SEQUENCE LISTING (3 sequences)

SEQ ID NO: 1 - protein, 37 aa, Artificial Sequence
  GLP-1 (1-37)
HDEFERHAEGTFTSDVSSYLEGQAAKEFIAWLVKGRG

SEQ ID NO: 2 - protein, 30 aa, Artificial Sequence
  GLP-1 (7-36)
  MOD_RES 30: R at position 30 is R-amide AMIDATION
HAEGTFTSDVSSYLEGQAAKEFIAWLVKGR

SEQ ID NO: 3 - protein, 31 aa, Artificial Sequence
  GLP-1 (7-37)
HAEGTFTSDVSSYLEGQAAKEFIAWLVKGRG

(19) United States
(12) Patent Application Publication
(10) Pub. No.: US 2024/0293514 A1
(43) Pub. Date: Sep. 5, 2024

(54) PHARMACEUTICAL COMPOSITIONS COMPRISING GLP-1R AGONISTS

(71) Applicants: 4MOVING BIOTECH, Lille (FR); SORBONNE UNIVERSITE, Paris (FR); ASSISTANCE PUBLIQUE HOPITAUX DE PARIS, Paris (FR); INSTITUT NATIONAL DE LA SANTÉ ET DE LA RECHERCHE MÉDICALE, Paris (FR)

(72) Inventors: Revital RATTENBACH, Lille (FR); Francis BERENBAUM, Lille (FR); Keren BISMUTH, Lille (FR); Céline MARTIN, Lille (FR); Coralie MEUROT, Lille (FR)

(21) Appl. No.: 18/573,737

(22) PCT Filed: Jun. 23, 2022
(86) PCT No.: PCT/EP2022/067269

§ 371 Date: Dec. 22, 2023

(30) Foreign Application Priority Data
Jun. 23, 2021 (EP) 21305865.4
Oct. 21, 2021 (EP) 21306467.8

(51) Int. Cl. A61K 38/26; A61K 9/00; A61K 47/10; A61K 47/18; A61K 47/26; A61P 19/02

(52) U.S. Cl. CPC A61K 38/26; A61K 9/0019; A61K 47/10; A61K 47/18; A61K 47/26; A61P 19/02

(57) ABSTRACT

The present invention relates to a pharmaceutical composition comprising a GLP-1R agonist such as liraglutide or semaglutide, a buffer selected from a tromethamine buffer and a phosphate buffer, and an isotonic agent selected from glucose, a polyethylene glycol and glycerol. The present invention also relates to said pharmaceutical composition for use in a method for treating joint diseases.

### FIELD OF INVENTION

The present invention relates to pharmaceutical compositions comprising GLP-1R agonists such as liraglutide or semaglutide. The present invention also relates to pharmaceutical compositions comprising GLP-1R agonists such as liraglutide or semaglutide for use in the treatment of joint diseases, the pharmaceutical compositions being administered, for example, via intraarticular injection.

### BACKGROUND OF INVENTION

Glucagon Like Peptide-1 (GLP-1) is a peptide hormone that binds to GLP-1 receptors expressed on the pancreatic beta cells, thus increasing the glucose transporter 2 expression and the secretion of insulin in response to increased blood glucose concentration. Moreover, GLP-1 reduces the secretion of some proinflammatory cytokines. GLP-1R agonists are commonly used as a treatment for type 2 diabetes.

Among the chronic joint diseases, osteoarthritis (OA) is the most prevalent disease, affecting nearly 50% of people over the age of 65 and occurring in younger people in case of anatomical abnormality, following a joint injury or in case of obesity. Worldwide, about 250 million people suffer from OA; this disease has major economic and social impacts.

Recently, it has been found that GLP-1R agonists such as liraglutide target relevant mechanism associated with inflammatory, antidegradative and regenerative processes relevant to OA. WO2020104833 thus relates to pharmaceutical compositions comprising GLP-1R agonists such as liraglutide, for use in the treatment of joint diseases, for example OA. The pharmaceutical compositions according to WO2020104833 are in particular in the form of gels that comprise liraglutide and albumin.

However, there is a need to find new formulations comprising GLP-1R agonists such as liraglutide or semaglutide that would be at least as effective as the formulations already on the market, while being able to induce a long-term effect when administered via intraarticular injection, in particular an effect that would last at least three weeks, more particularly an effect that would last at least four weeks. Indeed, limiting the frequency of intra-articular injections would both simplify the treatment for the patient and improve his or her comfort, and limit the number of medical procedures requiring the intervention of a caregiver.

The inventors have surprisingly found that the formulations of pharmaceutical compositions comprising GLP-1R agonists such as liraglutide or semaglutide according to the present invention are able to induce a long-term effect when administered via intraarticular injection, in particular an effect that lasts at least three weeks, more particularly an effect that lasts at least four weeks.

### SUMMARY

The present invention relates to a pharmaceutical composition for use in a method for treating joint diseases, in particular osteoarthritis and/or joint pain, more particularly inflammatory joint pain, wherein said pharmaceutical composition is a liquid phase to be administered via intraarticular injection, in particular via intraarticular injection into the joint cavity, and wherein said pharmaceutical composition comprises:
a GLP-1R agonist,
a buffer selected from the group consisting of a tromethamine buffer and a phosphate buffer, and
an isotonic agent selected from the group consisting of glucose, a polyethylene glycol, propylene glycol and glycerol.

Advantageously, the liquid phase may be selected from the group consisting of a solution, a suspension and an emulsion.

The present invention relates to a pharmaceutical composition for use in a method for treating joint diseases, in particular osteoarthritis and/or joint pain, more particularly inflammatory joint pain,
wherein said pharmaceutical composition is a solution or a suspension to be administered via intraarticular injection, in particular via intraarticular injection into the joint cavity, and
wherein said pharmaceutical composition comprises:
   a GLP-1R agonist,
   a buffer selected from the group consisting of a tromethamine buffer and a phosphate buffer, and
   an isotonic agent selected from the group consisting of glucose, a polyethylene glycol, propylene glycol and glycerol.

In one embodiment, the pharmaceutical composition is a solution.

In another embodiment, the pharmaceutical composition is a suspension.

Advantageously:
the GLP-1R agonist is selected from the group consisting of liraglutide, exenatide, lixisenatide, albiglutide, beinaglutide, dulaglutide, semaglutide, pegapamodutide, taspoglutide and combinations thereof; preferably, the GLP-1R agonist is selected from the group consisting of liraglutide, exenatide, lixisenatide, dulaglutide, semaglutide and combinations thereof; more preferably, the GLP-1R agonist is selected from the group consisting of liraglutide, semaglutide and combinations thereof; even more preferably, the GLP-1R agonist is liraglutide;
the buffer is a phosphate buffer, preferably a phosphate buffer comprising disodium phosphate dihydrate as buffering agent, and
the isotonic agent is propylene glycol.

Advantageously, the GLP-1R agonist is liraglutide or semaglutide, the buffer is a phosphate buffer, preferably a phosphate buffer comprising disodium phosphate dihydrate as buffering agent, and the isotonic agent is propylene glycol.

More advantageously, the GLP-1R agonist is liraglutide, the buffer is a phosphate buffer, preferably a phosphate buffer comprising disodium phosphate dihydrate as buffering agent, and the isotonic agent is propylene glycol.

More advantageously, the GLP-1R agonist is semaglutide, the buffer is a phosphate buffer, preferably a phosphate buffer comprising disodium phosphate dihydrate as buffering agent, and the isotonic agent is propylene glycol.

Advantageously, said pharmaceutical composition comprises:
a GLP-1R agonist, preferably the GLP-1R agonist is selected from the group consisting of liraglutide, exenatide, lixisenatide, albiglutide, beinaglutide, dulaglutide, semaglutide, pegapamodutide, taspoglutide and combinations thereof, more preferably the GLP-1R agonist is selected from the group consisting of liraglutide, exenatide, lixisenatide, dulaglutide, semaglutide and combinations thereof, even more preferably the GLP-1R agonist is selected from the group consisting of liraglutide, semaglutide and combinations thereof, better the GLP-1R agonist is liraglutide,
a buffer selected from the group consisting of a tromethamine buffer and a phosphate buffer, and
an isotonic agent selected from the group consisting of glucose, a polyethylene glycol and glycerol.

Advantageously, said pharmaceutical composition comprises:
a GLP-1R agonist, preferably the GLP-1R agonist is liraglutide or semaglutide,
a buffer selected from the group consisting of a tromethamine buffer and a phosphate buffer, and
an isotonic agent selected from the group consisting of glucose, a polyethylene glycol and glycerol.

Advantageously, said pharmaceutical composition comprises:
a GLP-1R agonist, preferably the GLP-1R agonist is liraglutide,
a buffer selected from the group consisting of a tromethamine buffer and a phosphate buffer, and
an isotonic agent selected from the group consisting of glucose, a polyethylene glycol and glycerol.

Advantageously, said pharmaceutical composition comprises:
a GLP-1R agonist, preferably the GLP-1R agonist is semaglutide,
a buffer selected from the group consisting of a tromethamine buffer and a phosphate buffer, and
an isotonic agent selected from the group consisting of glucose, a polyethylene glycol and glycerol.

Advantageously, the GLP-1R agonist is selected from the group consisting of liraglutide, exenatide, lixisenatide, albiglutide, beinaglutide, dulaglutide, semaglutide, pegapamodutide, taspoglutide and combinations thereof, preferably the GLP-1R agonist is selected from the group consisting of liraglutide, exenatide, lixisenatide, dulaglutide, semaglutide and combinations thereof, more preferably the GLP-1R agonist is selected from the group consisting of liraglutide, semaglutide and combinations thereof, even more preferably the GLP-1R agonist is liraglutide.

More advantageously, the GLP-1R agonist is liraglutide. Even more advantageously, the GLP-1R agonist is liraglutide and said pharmaceutical composition is to be administered at a dose from 0.0245 mg to 6.3 mg of liraglutide, preferably at a dose from 0.7 mg to 6.3 mg of liraglutide.

More advantageously, the GLP-1R agonist is liraglutide. Even more advantageously, the GLP-1R agonist is liraglutide and said pharmaceutical composition is to be administered at a dose from 0.0245 mg to 6.3 mg of liraglutide, preferably at a dose of 0.3 mg, 1.0 mg, 3.0 mg or 6.0 mg, of liraglutide.

More advantageously, the GLP-1R agonist is semaglutide. Even more advantageously, the GLP-1R agonist is semaglutide and said pharmaceutical composition is to be administered at a dose from 0.0245 mg to 6.3 mg of semaglutide, preferably at a dose from 0.7 mg to 6.3 mg of semaglutide, more preferably at a dose of 0.25 mg, 0.5 mg or 1 mg, of semaglutide.

More advantageously, the GLP-1R agonist is exenatide.

More advantageously, the GLP-1R agonist is lixisenatide.

More advantageously, the GLP-1R agonist is albiglutide.

More advantageously, the GLP-1R agonist is beinaglutide.

More advantageously, the GLP-1R agonist is dulaglutide.

More advantageously, the GLP-1R agonist is pegapamodutide.

More advantageously, the GLP-1R agonist is taspoglutide.

Advantageously, a dose of said pharmaceutical composition is to be administered in one or at least two intraarticular injections.

Preferably, doses of said pharmaceutical composition are to be administered every month.

Advantageously, the total dose of GLP-1R agonist that is administered in one year is from 0.18 mg to 72 mg, preferably from 0.7 mg to 8.4 mg.

The present invention also relates to a pharmaceutical composition, wherein said pharmaceutical composition is a liquid phase and comprises:
a GLP-1R agonist,
a buffer selected from the group consisting of a tromethamine buffer and a phosphate buffer, and
an isotonic agent selected from the group consisting of glucose, a polyethylene glycol and glycerol.

Advantageously, the liquid phase may be selected from the group consisting of a solution, a suspension and an emulsion.

The present invention also relates to a pharmaceutical composition, wherein said pharmaceutical composition is a solution or a suspension and comprises:
a GLP-1R agonist,
a buffer selected from the group consisting of a tromethamine buffer and a phosphate buffer, and
an isotonic agent selected from the group consisting of glucose, a polyethylene glycol and glycerol.

In one embodiment, the pharmaceutical composition is a solution.

In another embodiment, the pharmaceutical composition is a suspension.

Advantageously, the GLP-1R agonist is selected from the group consisting of liraglutide, exenatide, lixisenatide, albiglutide, beinaglutide, dulaglutide, semaglutide, pegapamodutide, taspoglutide and combinations thereof, preferably the GLP-1R agonist is selected from the group consisting of liraglutide, exenatide, lixisenatide, dulaglutide, semaglutide and combinations thereof, more preferably the GLP-1R agonist is selected from the group consisting of liraglutide, semaglutide and combinations thereof, even more preferably the GLP-1R agonist is liraglutide.

More advantageously, the GLP-1R agonist is liraglutide or semaglutide.

Even more advantageously, the GLP-1R agonist is semaglutide.

Even more advantageously, the GLP-1R agonist is liraglutide.

Even more advantageously, the GLP-1R agonist is exenatide.

Even more advantageously, the GLP-1R agonist is lixisenatide.

Even more advantageously, the GLP-1R agonist is albiglutide.

Even more advantageously, the GLP-1R agonist is beinaglutide.

Even more advantageously, the GLP-1R agonist is dulaglutide.

Even more advantageously, the GLP-1R agonist is pegapamodutide.

Even more advantageously, the GLP-1R agonist is taspoglutide.

Advantageously, the pharmaceutical composition comprises from 2 mg/mL to 20 mg/mL, preferably from 4 mg/mL to 8 mg/mL, more preferably about 6 mg/mL, of GLP-1R agonist.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist.

Advantageously, the buffer is a tromethamine buffer comprising tromethamine as buffering agent, preferably the pharmaceutical composition comprises from 0.1 mg/mL to 10 mg/mL, more preferably from 0.5 mg/mL to 1 mg/mL, even more preferably about 0.97 mg/mL, of tromethamine.

Advantageously, the buffer is a phosphate buffer comprising disodium phosphate as buffering agent, preferably the pharmaceutical composition comprises from 0.1 mg/mL to 10 mg/mL, more preferably from 0.75 mg/mL to 1.5 mg/mL, even more preferably about 1.14 mg/mL, of disodium phosphate.

Advantageously, the isotonic agent is glucose, preferably said pharmaceutical composition comprises from 10 mg/mL to 50 mg/mL, more preferably from 20 mg/mL to 40 mg/mL, even more preferably about 30 mg/mL, of glucose.

Advantageously, the isotonic agent is a polyethylene glycol having a molecular weight being less than 800 g·mol⁻¹, preferably from 100 g·mol⁻¹ to 600 g·mol-1, preferably the isotonic agent is PEG400. Preferably, said pharmaceutical composition comprises from 20 mg/mL to 100 mg/mL, more preferably from 40 mg/mL to 80 mg/mL, even more preferably about 60 mg/mL, of polyethylene glycol.

Advantageously, the isotonic agent is glycerol, preferably said pharmaceutical composition comprises from 5 mg/mL to 50 mg/mL, preferably from 10 mg/mL to 25 mg/mL, more preferably about 17 mg/mL or about 18 mg/mL, of glycerol.

### Definitions

In the present invention, the following terms have the following meanings:

“About”, before a figure or number, refers to plus or minus 10% of the face value of that figure or number. In one embodiment, “about”, before a figure or number, refers to plus or minus 5% of the face value of that figure or number.

“Active agent” refers to an agent that has a therapeutic effect. The agent may be a chemical or a biological substance. The therapeutic effect may be the prevention, delay, reduction in severity and/or frequency or suppression of at least one symptom associated with a pathological condition, or the prevention, slowing down or suppression of the underlying cause of a pathological condition, or the improvement or repair of a damage.

“Acute disease” refers to a non-chronic disease.

“Administration in combination” refers to sequential, simultaneous or separate administration of at least two active agents. When the administration in combination is simultaneous, the active agents administered simultaneously may be present in the same pharmaceutical composition.

“Buffer” refers to a mixture of a weak acid and its conjugate base, or a weak base and its conjugate acid, whose pH is maintained constant when a small amount of strong acid or base is added to it. In an embodiment, the buffer is a phosphate buffer. In an embodiment, the buffer is a tromethamine buffer. A “phosphate buffer” is a buffer which comprises phosphate or a derivative thereof as buffering agent. A “tromethamine buffer” is a buffer which comprises tromethamine or a derivative thereof as buffering agent.

“Buffering agent” refers to the specific chemical that is present in both an acidic and a basic forms in a buffer, allowing the pH of a pharmaceutical composition to be maintained constant. In an embodiment, the buffering agent is phosphate or a derivative thereof. In an embodiment, the buffering agent is tromethamine or a derivative thereof.

“Cartilage” or “cartilage matrix” or “articular cartilage” refers to elastic, translucent connective tissue in mammals, including human. Cartilage comprises chondrocytes, type II collagen, small amounts of other collagen types, other noncollagenous proteins, proteoglycans and water. Although most cartilage becomes bone upon maturation, some cartilage remains in its original form in some locations, such as the nose, ears, knees. The cartilage has no blood or nerve supply.

“Chronic disease” refers to a long-term, progressive illness, often associated with disability and the threat of serious complications. Chronic diseases evolve more or less rapidly for at least several months, in particular at least 3 months.

“Complex of GLP-1R agonist” refers to a polyatomic structure consisting of one or more independent entities (ions or molecules), in interaction, said structure comprising a GLP-1R agonist.

“Comprising” or “comprise” is to be construed in an open, inclusive sense, but not limited to. In an embodiment, “comprising” means “consisting essentially of”. In an embodiment, “comprising” means “consisting of”, which is to be construed as limited to.

“Dose” refers to the cumulative amount of GLP-1R agonists administered in 2 weeks to 1 month. In one embodiment, one “dose” refers to the cumulative amount of GLP-1R agonists administered in 2 weeks. In another embodiment, one “dose” refers to the cumulative amount of GLP-1R agonists administered in 3 weeks. In another embodiment, one “dose” refers to the cumulative amount of GLP-1R agonists administered in 1 month.

“Effective amount” of an active agent refers to a nontoxic but sufficient amount of said active agent to provide the desired therapeutic effect.

“Excipients” refers to any inactive ingredient, which is required for the formulation of an active agent in a suitable dosage form. In one embodiment, “excipients” refers to any and all solvents, diluents carriers, fillers, bulking agents, binders, disintegrants, polymer, lubricant, glidant, surfactants, isotonic agents, thickening or emulsifying agents, stabilizers, absorption accelerators, flavoring agents, preservatives, antioxidants, buffering agents, or any combination thereof. The person skilled in the art knows how to choose suitable excipients to obtain a formulation suitable for intra-articular injection, particularly in terms of viscosity, solvent, etc.

“From X to Y” refers to the range of values between X and Y, the limits X and Y being included in said range.

“Gel” refers to a non-fluid colloidal network or polymer network that is expanded throughout its whole volume by a fluid, the fluid being called “swelling agent”. “Hydrogel” refers to a gel in which the swelling agent is water. “Colloidal” refers to a state of subdivision, implying that the molecules or polymolecular particles dispersed in a medium have at least in one direction a dimension roughly from 1 nm to 1 μm. “Network” refers to a highly ramified structure in which essentially each constitutional unit is connected to each other constitutional unit and to the macroscopic phase boundary by many paths through the structure, the number of such paths increasing with the average number of intervening constitutional units; the paths must on average be co-extensive with the structure.

“GLP-1” or “glucagon-like peptide 1” refers to a 30- or 31-amino acid long peptide hormone deriving from the post-translational processing of the proglucagon peptide into “GLP-1 (1-37)”, which is further N-terminally truncated by tissue-specific post-translational processing in the intestinal L cells resulting in the two truncated and equipotent biologically active forms, “GLP-1 (7-36) amide” and “GLP-1 (7-37)”. In humans, GLP-1 (1-37) has an amino acid sequence as set forth in SEQ ID NO: 1; GLP-1 (7-36) amide has an amino acid sequence as set forth in SEQ ID NO: 2; and GLP-1 (7-37) has an amino acid sequence as set forth in SEQ ID NO: 3.

“GLP-1R agonists” or “GLP-1 receptor agonists” refers to agonists of the GLP-1 receptor (GLP1R). GLP-1R agonists may be GLP-1 analogues. GLP-1R agonists may be selected from the group consisting of liraglutide, exenatide, lixisenatide, albiglutide, beinaglutide, dulaglutide, semaglutide, pegapamodutide and taspoglutide. According to the present invention, the term “GLP-1G agonists” includes GLP-1R agonists enantiomers, GLP-1R agonists esters, GLP-1R agonists racemates, salts of GLP-1R agonists, solvates of GLP-1R agonists, hydrates of GLP-1R agonists, polymorphs of GLP-1R agonists and complexes of GLP-1R agonists.

“GLP-1R agonist enantiomer” refers to a molecule that has the same molecular formula and sequence of bonded atoms as a GLP-1R agonist, but differs from said GLP-1R agonist in the three-dimensional orientation of its atoms in space, the GLP-1R agonist and its enantiomer being mirror images of each other and non-superposable.

“GLP-1R agonist ester” refers to a GLP-1R agonist bonded to another chemical molecule via an ester group.

“GLP-1R agonist racemate” refers to a mixture in equal proportions of the levorotatory and dextrorotatory enantiomers of the GLP-1R agonist.

“Hydrate of a compound” refers to a molecular complex comprising the compound and one or more pharmaceutically acceptable solvent molecules, wherein the solvent is water.

“Intraarticular injection” refers to an injection directly into the closed cavity of a joint in the human body.

“Isotonic agent” refers to an agent added to a pharmaceutical composition to ensure isotonicity between the pharmaceutical composition and the biological medium in which the pharmaceutical composition is administered.

“Joint” and “articulation” are used interchangeably.

“Joint disease” refers to any disease affecting at least one joint in a human body. Examples of joint diseases include, but are not limited to, inflammatory arthritis (in particular osteoarthritis, rheumatoid arthritis, psoriatic arthritis, juvenile arthritis, ankylosing spondylitis, lupus and related connective tissue diseases, synovitis crystal arthropathies (gout, chondrocalcinosis, oochronsis, hydroxyapatitis), abarticular pathologies (tendinitis, capsulitis, enthesitis), septic arthritis, subchondral bone pathologies (osteonecrosis, insufficiency fracture, bone marrow lesions), genetic arthropathies, inflammatory joint pain or any other joint pains. Joint diseases can be characterized by one or several symptoms including, without limitation, limitation of motion, joint pain, joint inflammation, joint tenderness, joint stiffness, and joint swelling.

“Liraglutide” refers to a 32-amino acid peptide of 3.7 kDa. It is a synthetic acylated analog of human GLP-1 (7-37), in which the lysine residue at position 28 (SEQ ID NO: 3 numbering) is replaced by an arginine residue, and a C16 fatty acid (palmitic acid) is bound to the ε-amino group of the lysine residue at position 20 (SEQ ID NO: 3 numbering) through a γ-glutamyl linker. The chemical structure of liraglutide is:

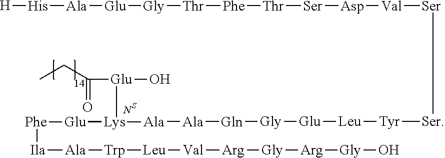

“Osteoarthritis” or “OA” is a joint disease. Osteoarthritis is a disorder that can affect any moveable joint of the body, for example knees, hips, and/or hands. It can show itself as a breakdown of tissues and abnormal changes to cell structures of joints, which can be initiated by injury. As the joint tries to repair, it can lead to other problems. Osteoarthritis first shows itself as a change to the biological processes within a joint, followed by abnormal changes to the joint, such as the breakdown of cartilage, bone reshaping, bony lumps, joint inflammation, and loss of joint function. This can result in pain, stiffness and loss of movement. There are certain factors which make some people more vulnerable to developing osteoarthritis, such as genetic factors, other joint disorders (such as rheumatoid arthritis), injury to the joint from accidents or surgery, being overweight or doing heavy physical activity in some sports or a person's job.

“Pharmaceutical composition” refers to the combination of at least one active agent and at least one pharmaceutically acceptable excipient.

“Pharmaceutically acceptable” refers to generally safe, non-toxic and neither biologically nor physiologically nor otherwise undesirable for animals, in particular for humans.

“Polymorph of GLP-1R agonist” refers to another crystal structure of said GLP-1R agonist.

“Preservative” refers to any substance or chemical that is added to a composition to prevent its decomposition by microbial growth or by undesirable chemical changes.

“Salts of GLP-1R agonist” refers to acid or base addition salts of GLP-1R agonist. The acid addition salts are formed with pharmaceutically acceptable organic or inorganic acids; the base addition salts are formed when an acid proton present in the GLP-1R agonist is either replaced by a metal ion or coordinated with a pharmaceutically acceptable organic or inorganic base.

“Solution” refers to a liquid homogeneous phase comprising at least one solvent in which at least one solute is dissolved, the at least one solute being the minor component of the solution. According to the present invention, a solution is not a gel, and thus does not comprise a non-fluid colloidal network or polymer network. Advantageously, a solution does not comprise a polymer selected from the group consisting of non-ionic surfactant, cellulose, polyether, glucan, glycerophospholipids, polysaccharides, proteins, and combinations thereof.

“Solvate of GLP-1R agonist” refers to a molecular complex comprising a GLP-1R agonist and one or more pharmaceutically acceptable solvent molecules. “Hydrate of GLP-1R agonist” refers to a molecular complex comprising a GLP-1R agonist and one or more pharmaceutically acceptable solvent molecules, wherein the solvent is water.

“Subject” or “patient” refers to an animal, in particular a mammal. In one embodiment, “subject” refers to an animal selected from the group consisting of a dog, a cat, a horse, a cow, a sheep, a goat and a non-human primate. In one preferred embodiment, “subject” refers to a human (man or woman). According to a preferred embodiment, “subject” refers to a human over the age of 18, preferably over the age of 50, more preferably over the age of 65.

“Suspension” refers to a liquid homogeneous phase comprising at least one solvent in which at least one solute is dispersed, the at least one solute being solid particles and being the minor component of the solution. According to the present invention, a suspension is not a gel, and thus does not comprise a non-fluid colloidal network or polymer network. Advantageously, a suspension does not comprise a polymer selected from the group consisting of non-ionic surfactant, cellulose, polyether, glucan, glycerophospholipids, polysaccharides, proteins, and combinations thereof.

“Therapeutically effective amount” of an active agent refers to a nontoxic but sufficient amount of said active agent to provide the desired therapeutic effect.

“Treating” or “treatment” refers to any action which makes it possible to prevent, delay, reduce in severity and/or frequency or suppress at least one symptom associated with a pathological condition, or to prevent, slow down or suppress the underlying cause of a pathological condition, or the improvement or remediation of damage. In particular, in the context of the present invention, the term “treating” or “treatment” may refer more particularly to the inhibition or the slowing down of the arthritic destruction of cartilage. In particular, in the context of the present invention, the term “treating” or “treatment” may refer more particularly to the reduction or even the suppression of a joint pain. In one embodiment, “treatment” refers to a curative treatment. In another embodiment, “treatment” refers to a preventive treatment. In another embodiment, “treatment” refers to a preventive and/or curative treatment.

“Water for injection” is a water intended either for the preparation of parenteral drug with an aqueous vehicle (bulk water for injection) or for the dissolution or dilution of active agents or preparations for parenteral administration (sterilized water for injection).

### DETAILED DESCRIPTION

### Pharmaceutical Composition

This invention relates to a pharmaceutical composition comprising at least one GLP-1R agonist or a pharmaceutically acceptable ester, salt, complex, polymorph, hydrate, solvate, enantiomer or racemate thereof, a buffer and at least one isotonic agent.

As mentioned in the above definitions, the mention of a GLP-1R agonist in the present invention also encompasses any pharmaceutically acceptable ester, salt, complex, polymorph, hydrate, solvate, enantiomer or racemate of said GLP1-R agonist.

The pharmaceutical composition of the invention comprises a GLP1-R agonist as active agent, and a mixture of excipients preferably suitable for intraarticular injection, said mixture of excipients comprising a buffer and at least one isotonic agent.

The pharmaceutical composition according to the invention is a solution or a suspension. The solvent of the pharmaceutical composition according to the invention may advantageously be water, more advantageously water for injection.

### Detailed Compounds of the Pharmaceutical Composition

Advantageously, the GLP-1R agonist is selected from the group consisting of a polypeptide, an antibody, a nucleic acid, an aptamer, and a small molecule. Preferably, the GLP-1R agonist is a polypeptide. The polypeptide may be selected from the group consisting of liraglutide, exenatide, lixisenatide, albiglutide, beinaglutide, dulaglutide, semaglutide, and taspoglutide. In a preferred embodiment, the GLP-1R agonist is liraglutide or semaglutide. In a more preferred embodiment, the GLP-1R agonist is liraglutide. In another more preferred embodiment, the GLP-1R agonist is semaglutide.

In one embodiment, the GLP-1R agonist is liraglutide.

In another embodiment, the GLP-1R agonist is exenatide.

In another embodiment, the GLP-1R agonist is lixisenatide.

In another embodiment, the GLP-1R agonist is albiglutide.

In another embodiment, the GLP-1R agonist is beinaglutide.

In another embodiment, the GLP-1R agonist is dulaglutide.

In another embodiment, the GLP-1R agonist is semaglutide.

In another embodiment, the GLP-1R agonist is pegapamodutide.

In another embodiment, the GLP-1R agonist is taspoglutide.

Advantageously, the buffer is selected from the group consisting of a tromethamine buffer, a phosphate buffer and any combination thereof.

Advantageously, the buffer is a tromethamine buffer. The tromethamine buffer may comprise a buffering agent selected from the group consisting of tromethamine (Tris), tromethamine (Tris) acetate, tromethamine (Tris) phosphate, and any combination thereof. More preferably, the buffer is a tromethamine buffer comprising tromethamine (Tris) as buffering agent.

Advantageously, the buffer is a phosphate buffer. The phosphate buffer may comprise a buffering agent selected from the group consisting of dibasic calcium phosphate, tribasic calcium phosphate, monobasic potassium phosphate, dibasic potassium phosphate, monobasic sodium phosphate, disodium phosphate and any hydrate or combination thereof. More preferably, the buffer is a phosphate buffer comprising disodium phosphate or disodium phosphate dihydrate as buffering agent. Even more preferably, the buffer is phosphate-buffered saline (PBS).

Advantageously, the isotonic agent is selected from the group consisting of glucose, a polyethylene glycol, propylene glycol, glycerol and any combination thereof.

Advantageously, the isotonic agent is selected from the group consisting of glucose, a polyethylene glycol, glycerol and any combination thereof.

Advantageously, the isotonic agent is propylene glycol.

Advantageously, the isotonic agent is a polyethylene glycol. The polyethylene glycol may be a polyethylene glycol having a molecular weight less than 800 g·mol⁻¹, preferably from 100 g·mol⁻¹ to 800 g·mol⁻¹, more preferably from 100 g·mol⁻¹ to 600 g·mol⁻¹, even more preferably a polyethylene glycol having a molecular weight from 200 g·mol⁻¹ to 400 g·mol⁻¹, even more preferably a polyethylene glycol having a molecular weight of 400 g·mol⁻¹ (=PEG400) or of 200 g·mol⁻¹ (=PEG200). In one embodiment, the polyethylene glycol is a polyethylene glycol having a molecular weight of 100 g·mol⁻¹, 200 g·mol⁻¹, 300 g·mol⁻¹, 400 g·mol⁻¹, 500 g·mol⁻¹, 600 g·mol⁻¹, 700 g·mol⁻¹ or 800 g·mol⁻¹.

Advantageously, the pharmaceutical composition comprises:
a GLP-1R agonist,
a buffer selected from the group consisting of a tromethamine buffer and a phosphate buffer, and
an isotonic agent selected from the group consisting of glucose, a polyethylene glycol, propylene glycol and glycerol.

Advantageously, the pharmaceutical composition comprises:
a GLP-1R agonist,
a buffer selected from the group consisting of a tromethamine buffer and a phosphate buffer, and
an isotonic agent selected from the group consisting of glucose, a polyethylene glycol and glycerol.

Advantageously, the pharmaceutical composition comprises:
a GLP-1R agonist,
a tromethamine buffer, and
an isotonic agent selected from the group consisting of glucose, a polyethylene glycol and glycerol.

Advantageously, the pharmaceutical composition comprises:
a GLP-1R agonist,
a phosphate buffer, and
an isotonic agent selected from the group consisting of glucose and a polyethylene glycol.

Advantageously, the pharmaceutical composition comprises:
a GLP-1R agonist,
a buffer selected from the group consisting of a tromethamine buffer and a phosphate buffer, and
an isotonic agent selected from the group consisting of glucose, propylene glycol and glycerol.

Advantageously, the pharmaceutical composition comprises:
a GLP-1R agonist,
a tromethamine buffer, and
an isotonic agent selected from the group consisting of glucose, a polyethylene glycol, propylene glycol and glycerol.

Advantageously, the pharmaceutical composition comprises:
a GLP-1R agonist,
a tromethamine buffer, and
an isotonic agent selected from the group consisting of glucose, propylene glycol and glycerol.

Advantageously, the pharmaceutical composition comprises:
a GLP-1R agonist,
a buffer selected from the group consisting of a tromethamine buffer and a phosphate buffer, and
glucose.

Advantageously, the pharmaceutical composition comprises:
a GLP-1R agonist,
a tromethamine buffer, and
glucose.

In one preferred embodiment, the pharmaceutical composition comprises:
liraglutide or semaglutide,
a buffer selected from the group consisting of a tromethamine buffer and a phosphate buffer, and
an isotonic agent selected from the group consisting of glucose, a polyethylene glycol, propylene glycol and glycerol.

In one preferred embodiment, the pharmaceutical composition comprises:
liraglutide or semaglutide,
a tromethamine buffer, and
an isotonic agent selected from the group consisting of glucose, a polyethylene glycol, propylene glycol and glycerol.

In one preferred embodiment, the pharmaceutical composition comprises:
liraglutide or semaglutide,
a phosphate buffer, and
an isotonic agent selected from the group consisting of glucose, a polyethylene glycol, propylene glycol and glycerol.

In one preferred embodiment, the pharmaceutical composition comprises:
liraglutide,
a buffer selected from the group consisting of a tromethamine buffer and a phosphate buffer, and
an isotonic agent selected from the group consisting of glucose, a polyethylene glycol, propylene glycol and glycerol.

In one preferred embodiment, the pharmaceutical composition comprises:
liraglutide,
a tromethamine buffer, and
an isotonic agent selected from the group consisting of glucose, a polyethylene glycol, propylene glycol and glycerol.

In one preferred embodiment, the pharmaceutical composition comprises:
liraglutide,
a phosphate buffer, and
an isotonic agent selected from the group consisting of glucose, a polyethylene glycol, propylene glycol and glycerol.

In one preferred embodiment, the pharmaceutical composition comprises:
semaglutide,
a buffer selected from the group consisting of a tromethamine buffer and a phosphate buffer, and
an isotonic agent selected from the group consisting of glucose, a polyethylene glycol, propylene glycol and glycerol.

In one preferred embodiment, the pharmaceutical composition comprises:
semaglutide,
a tromethamine buffer, and
an isotonic agent selected from the group consisting of glucose, a polyethylene glycol, propylene glycol and glycerol.

In one preferred embodiment, the pharmaceutical composition comprises:
semaglutide,
a phosphate buffer, and
an isotonic agent selected from the group consisting of glucose, a polyethylene glycol, propylene glycol and glycerol.

Advantageously, the pharmaceutical composition comprises:
a GLP-1R agonist,
a buffer selected from the group consisting of a tromethamine buffer and a phosphate buffer, and
an isotonic agent selected from the group consisting of glucose, a polyethylene glycol and glycerol.

In one preferred embodiment, the pharmaceutical composition comprises:
liraglutide or semaglutide,
a buffer selected from the group consisting of a tromethamine buffer and a phosphate buffer, and
an isotonic agent selected from the group consisting of glucose, a polyethylene glycol and glycerol.

In one preferred embodiment, the pharmaceutical composition comprises:
liraglutide or semaglutide,
a tromethamine buffer, and
an isotonic agent selected from the group consisting of glucose, a polyethylene glycol and glycerol.

In one preferred embodiment, the pharmaceutical composition comprises:
liraglutide or semaglutide,
a phosphate buffer, and
an isotonic agent selected from the group consisting of glucose, a polyethylene glycol and glycerol.

In one preferred embodiment, the pharmaceutical composition comprises:
liraglutide or semaglutide,
a buffer selected from the group consisting of a tromethamine buffer and a phosphate buffer, and
glucose as isotonic agent.

In one preferred embodiment, the pharmaceutical composition comprises:
liraglutide or semaglutide,
a buffer selected from the group consisting of a tromethamine buffer and a phosphate buffer, and
a polyethylene glycol, preferably PEG400, as isotonic agent.

In one preferred embodiment, the pharmaceutical composition comprises:
liraglutide or semaglutide,
a buffer selected from the group consisting of a tromethamine buffer and a phosphate buffer, and
glycerol as isotonic agent.

In one preferred embodiment, the pharmaceutical composition comprises:
liraglutide or semaglutide,
a buffer selected from the group consisting of a tromethamine buffer and a phosphate buffer, and
propylene glycol as isotonic agent.

In one preferred embodiment, the pharmaceutical composition comprises:
liraglutide,
a buffer selected from the group consisting of a tromethamine buffer and a phosphate buffer, and
an isotonic agent selected from the group consisting of glucose, a polyethylene glycol and glycerol.

In one preferred embodiment, the pharmaceutical composition comprises:
liraglutide,
a tromethamine buffer, and
an isotonic agent selected from the group consisting of glucose, a polyethylene glycol and glycerol.

In one preferred embodiment, the pharmaceutical composition comprises:
liraglutide,
a phosphate buffer, and
an isotonic agent selected from the group consisting of glucose, a polyethylene glycol and glycerol.

In one preferred embodiment, the pharmaceutical composition comprises:
liraglutide,
a buffer selected from the group consisting of a tromethamine buffer and a phosphate buffer, and
glucose as isotonic agent.

In one preferred embodiment, the pharmaceutical composition comprises:
liraglutide,
a buffer selected from the group consisting of a tromethamine buffer and a phosphate buffer, and
a polyethylene glycol, preferably PEG400, as isotonic agent.

In one preferred embodiment, the pharmaceutical composition comprises:
liraglutide,
a buffer selected from the group consisting of a tromethamine buffer and a phosphate buffer, and
glycerol as isotonic agent.

In one preferred embodiment, the pharmaceutical composition comprises:
liraglutide,
a buffer selected from the group consisting of a tromethamine buffer and a phosphate buffer, and
propylene glycol as isotonic agent.

In one preferred embodiment, the pharmaceutical composition comprises:
semaglutide,
a buffer selected from the group consisting of a tromethamine buffer and a phosphate buffer, and
an isotonic agent selected from the group consisting of glucose, a polyethylene glycol and glycerol.

In one preferred embodiment, the pharmaceutical composition comprises:
semaglutide,
a tromethamine buffer, and
an isotonic agent selected from the group consisting of glucose, a polyethylene glycol and glycerol.

In one preferred embodiment, the pharmaceutical composition comprises:
semaglutide,
a phosphate buffer, and
an isotonic agent selected from the group consisting of glucose, a polyethylene glycol and glycerol.

In one preferred embodiment, the pharmaceutical composition comprises:
semaglutide,
a buffer selected from the group consisting of a tromethamine buffer and a phosphate buffer, and
glucose as isotonic agent.

In one preferred embodiment, the pharmaceutical composition comprises:
semaglutide,
a buffer selected from the group consisting of a tromethamine buffer and a phosphate buffer, and
a polyethylene glycol, preferably PEG400, as isotonic agent.

In one preferred embodiment, the pharmaceutical composition comprises:
semaglutide,
a buffer selected from the group consisting of a tromethamine buffer and a phosphate buffer, and
glycerol as isotonic agent.

In one preferred embodiment, the pharmaceutical composition comprises:
semaglutide,
a buffer selected from the group consisting of a tromethamine buffer and a phosphate buffer, and
propylene glycol as isotonic agent.

In one preferred embodiment, the pharmaceutical composition comprises:
a GLP-1R agonist,
a phosphate buffer, and
propylene glycol as isotonic agent.

In one preferred embodiment, the pharmaceutical composition comprises:
liraglutide or semaglutide,
a phosphate buffer, and
propylene glycol as isotonic agent.

In one preferred embodiment, the pharmaceutical composition comprises:
liraglutide or semaglutide,
a phosphate buffer,
propylene glycol as isotonic agent, and
phenol.

In one preferred embodiment, the pharmaceutical composition comprises:
liraglutide,
a phosphate buffer, and
propylene glycol as isotonic agent.

In one preferred embodiment, the pharmaceutical composition comprises:
liraglutide,
a phosphate buffer,
propylene glycol as isotonic agent, and
phenol.

In one preferred embodiment, the pharmaceutical composition comprises:
semaglutide,
a phosphate buffer, and
propylene glycol as isotonic agent.

In one preferred embodiment, the pharmaceutical composition comprises:
semaglutide,
a phosphate buffer,
propylene glycol as isotonic agent, and
phenol.

Advantageously, the pharmaceutical composition according to the invention does not comprise propylene glycol.

Advantageously, the pharmaceutical composition of the invention further comprises at least one additional excipient. The additional excipient may be a preservative. The preservative may be selected from the group consisting of phenol, cresol, resorcinol, parabens and any combination thereof. Preferably, the preservative is phenol.

Advantageously, the pharmaceutical composition of the invention comprises at least one further active agent used in the treatment of joint diseases, in particular of osteoarthritis. The further active agent used in the treatment of joint diseases, in particular of osteoarthritis, may be selected from the group consisting of incretins such as GIP (Glucose-dependent insulin releasing polypeptide), inhibitors of the dipeptidyl peptidase IV enzyme, growth factors or growth factors targeting agents (FGF-18, BMP7, anti-NGF agents), Wnt pathway molecules targeting agents (DYRK1A targeting agents, CLK2 targeting agents), metalloproteinases and/or aggrecanases targeting agents (ADAMTS4 targeting agents, ADAMTS5 targeting agents, MMPs targeting agents), senescence pathway targeting agents, bone resorption molecules targeting agents (cathepsin K targeting agents), analgesics (such as opioids, tramadol, acetaminophen, capsaicin), nonsteroidal anti-inflammatory drugs (such as modified angiopoietin-like 3 (ANGPTL3) protein (for example LNA043) and anti-IL1 (for example anakinra, canakinumab)), steroidal anti-inflammatory drugs, symptomatic slow-acting anti-arthritic agents (SYSADOAs), hyaluronic acids, platelet rich plasmas (PRPs), alpha-1 glycoprotein and albumin.

Advantageously, the pharmaceutical composition of the invention comprises at least one further active agent used in the treatment of joint diseases, in particular of osteoarthritis. The at least one further active agent used in the treatment of joint diseases, in particular of osteoarthritis, may be selected from the group consisting of incretins such as GIP (Glucose-dependent insulin releasing polypeptide), inhibitors of the dipeptidyl peptidase IV enzyme, growth factors or growth factors targeting agents (FGF-18, BMP7, anti-NGF agents), Wnt pathway molecules targeting agents (DYRK1A targeting agents, CLK2 targeting agents), metalloproteinases and/or aggrecanases targeting agents (ADAMTS4 targeting agents, ADAMTS5 targeting agents, MMPs targeting agents), senescence pathway targeting agents, bone resorption molecules targeting agents (cathepsin K targeting agents), analgesics (such as opioids, tramadol, acetaminophen, capsaicin), nonsteroidal anti-inflammatory drugs (such as ibuprofen, ketoprofen, diclofenac, celecoxib, indomethacin), anti-arthritic (such as modified angiopoietin-like 3 (ANGPTL3) protein (for example LNA043)), anti-IL1 (for example anakinra, canakinumab)), steroidal anti-inflammatory drugs, symptomatic slow-acting anti-arthritic agents (SYSADOAs), hyaluronic acids, platelet rich plasmas (PRPs), alpha-1 glycoprotein, albumin and cellular therapies (such as injection of stem cells and Mesenchymal Stromal Cells).

The Glucose-dependent insulin releasing polypeptide may be selected from the group consisting of GIP receptor antagonist (for example anti-GIPR monoclonal antibody from Amgen) and tirzepatide (from Lilly).

The inhibitor of the dipeptidyl peptidase IV enzyme may be selected from the group consisting of sitagliptin, saxagliptin, vildagliptin, alogliptin and linagliptin.

The growth factors may be selected from the group consisting of fibroblast growth factor (FGF-18 sprifermin), NGF and BMP7 protein.

The growth factors targeting agents may be selected from the group consisting of fibroblast growth factor targeting agents, anti-NGF agents such as tanezumab and BMP7 protein targeting agents.

The Wnt pathway molecules targeting may be selected from the group consisting of CLK2 inhibitors, DYRK1A inhibitors and lorecivivint.

Metalloproteinases and aggrecanases may be selected from the group consisting of ADAMTS5 inhibitors and ADAMTS5 antibodies.

Senescence pathway targeting may be MDM2-p53 interaction inhibitors.

The bone resorption molecules may be cathepsin K.

The analgesics may be selected from the group consisting of acetylsalicylic acid, lysine acetylsalicylate, phenylbutazone, sulindac, diclofenac potassium or sodium, aceclofenac, tiaprofenic acid, ibuprofen, ketoprofen, alminoprofen, fenoprofen, naproxen, flurbiprofen, indomethacin, mefenamic acid, niflumic acid, tenoxicam, meloxicam, piroxicam, celecoxib, etoricoxib, betamethasone, dexamethasone, prednisone, prednisolone, tixocortol, triamcinolone, CNTX-4975 and bedinvetmab.

The nonsteroidal anti-inflammatory drugs may be selected from the group consisting of acetylsalicylic acid, lysine acetylsalicylate, phenylbutazone, sulindac, diclofenac potassium or sodium, aceclofenac, tiaprofenic acid, ibuprofen, ketoprofen, alminoprofen, fenoprofen, naproxen, flurbiprofen, indomethacin, mefenamic acid, niflumic acid, tenoxicam, meloxicam, piroxicam, celecoxib and etoricoxib.

The steroidal anti-inflammatory drugs may be selected from the group consisting of betamethasone, dexamethasone, prednisone, prednisolone, tixocortol and triamcinolone.

The symptomatic slow-acting anti-arthritic agents may be selected from the group consisting of chondroitin, chondroitin sulphate, glucosamine, glucosamine sulphate, diacerein, and unsaponifiable extracts of avocado and soya (such as in the marketed product Piascledine®).

According to an embodiment, the at least one further active agent used in the treatment of joint diseases is selected from the group consisting of hyaluronic acid, albumin and alpha-1 glycoprotein.

In one embodiment, the pharmaceutical composition is a solution. The solution may be an aqueous solution. The aqueous solution may be an aqueous solution for injection, even more particularly an aqueous solution for intra-articular injection.

In one embodiment, the pharmaceutical composition is a suspension. The solution may be an aqueous suspension. The aqueous suspension may be an aqueous suspension for injection, even more particularly an aqueous suspension for intra-articular injection.

### Concentrations of the Compounds of the Pharmaceutical Composition

Advantageously, the pharmaceutical composition comprises from 2 mg/mL to 20 mg/mL, preferably from 2 mg/mL to 8 mg/mL, more preferably from 4 mg/mL to 8 mg/mL, even more preferably about 6 mg/mL, of GLP-1R agonist. In one embodiment, the pharmaceutical composition comprises about 2, 3, 4, 5, 6, 7, 8, 9, 10, 11, 12, 13, 14, 15, 16, 17, 18, 19 or 20 mg/mL, of GLP-1R agonist. In one embodiment, the pharmaceutical composition comprises about 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist.

Advantageously, the pharmaceutical composition comprises from 0.01 mg/mL to 100 mg/mL, preferably from 0.5 mg/mL to 80 mg/mL, more preferably from 1 mg/mL to 60 mg/mL, even more preferably about 1.34 mg/mL, of GLP-1R agonist. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist.

Advantageously, the pharmaceutical composition comprises from 2 mg/mL to 20 mg/mL, preferably from 2 mg/mL to 8 mg/mL, more preferably from 4 mg/mL to 8 mg/mL, even more preferably about 6 mg/mL, of GLP-1R agonist, wherein the GLP-1R agonist is liraglutide. In one embodiment, the pharmaceutical composition comprises about 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is liraglutide.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist, wherein the GLP-1R agonist is semaglutide. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is semaglutide.

Advantageously, the pharmaceutical composition comprises from 0.01 mg/mL to 20 mg/mL, preferably from 0.5 mg/mL to 4 mg/mL, more preferably from 2.5 mg/mL to 3.5 mg/mL, even more preferably about 3.01 mg/mL, of GLP-1R agonist, wherein the GLP-1R agonist is exenatide. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is exenatide.

Advantageously, the pharmaceutical composition comprises from 0.01 mg/mL to 20 mg/mL, preferably from 0.01 mg/mL to 2 mg/mL, more preferably from 0.05 mg/mL to 1.0 mg/mL, even more preferably about 0.1 mg/mL, of GLP-1R agonist, wherein the GLP-1R agonist is lixisenatide. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is lixisenatide.

Advantageously, the pharmaceutical composition comprises from 0.01 mg/mL to 100 mg/mL, preferably from 10 mg/mL to 80 mg/mL, more preferably from 50 mg/mL to 70 mg/mL, even more preferably about 60 mg/mL, of GLP-1R agonist, wherein the GLP-1R agonist is albiglutide. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is albiglutide.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist, wherein the GLP-1R agonist is beinaglutide. In one embodiment, the pharmaceutical composition comprises about 0.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is beinaglutide.

Advantageously, the pharmaceutical composition comprises from 0.01 mg/mL to 20 mg/mL, preferably from 1.5 mg/mL to 9 mg/mL, more preferably from 3 mg/mL to 9 mg/mL, even more preferably about 9 mg/mL, of GLP-1R agonist, wherein the GLP-1R agonist is dulaglutide. In one embodiment, the pharmaceutical composition comprises about 00.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is dulaglutide.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist, wherein the GLP-1R agonist is pegapamodutide. In one embodiment, the pharmaceutical composition comprises about 00.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is pegapamodutide.

Advantageously, the pharmaceutical composition comprises from 0.01 mg/mL to 20 mg/mL, preferably from 0.5 mg/mL to 2 mg/mL, more preferably from 1 mg/mL to 1.5 mg/mL, even more preferably about 1.34 mg/mL, of GLP-1R agonist, wherein the GLP-1R agonist is taspoglutide. In one embodiment, the pharmaceutical composition comprises about 00.01, 0.1, 0.2, 0.3, 0.4, 0.5, 0.6, 0.61, 0.62, 0.63, 0.64, 0.65, 0.66, 0.67, 0.68, 0.69, 0.7, 0.8, 0.9, 1, 1.1, 1.2, 1.30, 1.31, 1.32, 1.33, 1.34, 1.35, 1.36, 1.37, 1.38, 1.39, 1.4, 1.5, 1.6, 1.7, 1.8, 1.9, 2, 3, 4, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12, 13, 14, 15, 16, 17, 18, 19 or 20 mg/mL, of GLP-1R agonist, wherein the GLP-1R agonist is taspoglutide.

Advantageously, the pharmaceutical composition comprises from 0.1 mg/mL to 10 mg/mL, more preferably from 0.5 mg/mL to 1 mg/mL, even more preferably about 0.97 mg/mL, of tromethamine. In one embodiment, the pharmaceutical composition comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/mL, of tromethamine.

Advantageously, the pharmaceutical composition comprises from 0.80 mM to 80 mM, preferably from 2 mM to 20 mM, more preferably from 6 mM to 10 mM, even more preferably about 8 mM, of tromethamine.

Advantageously, the pharmaceutical composition comprises from 0.1 mg/mL to 10 mg/mL, preferably from 0.75 mg/mL to 1.5 mg/mL, more preferably about 1.14 mg/mL, of disodium phosphate. In one embodiment, the pharmaceutical composition comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/mL, of disodium phosphate. In one embodiment, the pharmaceutical composition comprises from 0.70 mM to 70 mM, preferably from 2 mM to 20 mM, more preferably from 3 mM to 10 mM, even more preferably about 8 mM or about 3 mM, of disodium phosphate.

Advantageously, the pharmaceutical composition comprises from 0.05 mg/mL to 10 mg/mL, preferably from 0.25 mg/mL to 2.0 mg/mL, of disodium phosphate dihydrate. Preferably, the pharmaceutical composition comprises about 0.47 mg/mL of disodium phosphate dihydrate. More preferably, the pharmaceutical composition comprises about 1.42 mg/mL of disodium phosphate dihydrate. In one embodiment, the pharmaceutical composition comprises about 0.05, 0.1, 0.2, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.6, 0.7, 0.8, 0.9, 1.0, 1.1, 1.2, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49 1.50, 1.51, 1.52, 1.53, 1.54, 1.55, 1.56, 1.57, 1.58, 1.59, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/mL, of disodium phosphate dihydrate.

Advantageously, the pharmaceutical composition comprises from 10 mg/mL to 50 mg/mL, preferably from 20 mg/mL to 40 mg/mL, more preferably about 30 mg/mL, of glucose. In one embodiment, the pharmaceutical composition comprises about 10, 15, 20, 25, 26, 27, 28, 29, 30, 31, 32, 33, 34, 35, 40, 45 or 50 mg/mL, of glucose.

Advantageously, the pharmaceutical composition comprises from 20 mg/mL to 100 mg/mL, preferably from 40 mg/mL to 80 mg/mL, more preferably about 60 mg/mL, of a polyethylene glycol, particularly of PEG400 or PEG200, more particularly of PEG400. In one embodiment, the pharmaceutical composition comprises about 20, 25, 30, 35, 40, 45, 50, 55, 56, 57, 58, 59, 60, 61, 62, 63, 64, 65, 70, 75, 80, 85, 90, 95 or 100 mg/mL, of a polyethylene glycol, particularly of PEG400 or PEG200, more particularly of PEG400.

Advantageously, the pharmaceutical composition comprises from 5 mg/mL to 50 mg/mL, preferably from 10 mg/mL to 25 mg/mL, more preferably about 17 mg/mL or about 18 mg/mL, of glycerol. In one embodiment, the pharmaceutical composition comprises about 5, 10, 11, 12, 13, 14, 15, 16, 17, 18, 19, 20, 21, 22, 23, 24, 25, 26, 27, 28, 29, 30, 35, 40, 45 or 50 mg/mL, of glycerol.

Advantageously, the pharmaceutical composition comprises from 1 mg/mL to 20 mg/mL of propylene glycol. Preferably, the pharmaceutical composition comprises from 1 mg/mL to 10 mg/mL, more preferably from 4 mg/mL to 6 mg/mL, even more preferably about 4.7 mg/mL, of propylene glycol. Preferably, the pharmaceutical composition comprises from 10 mg/mL to 20 mg/mL, more preferably from 12 mg/mL to 16 mg/mL, even more preferably about 14 mg/mL, of propylene glycol. In one embodiment, the pharmaceutical composition comprises about 1, 2, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 8, 9, 10, 11, 12.0, 12.5, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.5, 16, 17, 18, 19 or 20 mg/mL, of propylene glycol.

Advantageously, the pharmaceutical composition comprises from 0.1 mg/mL to 10 mg/mL, preferably from 1 mg/mL to 7.5 mg/mL, of phenol. Preferably, the pharmaceutical composition comprises from 1 mg/mL to 2.5 mg/mL, more preferably about 1.8 mg/mL, of phenol. Preferably, the pharmaceutical composition comprises from 2.5 mg/mL to 7.5 mg/mL, more preferably about 5.5 mg/mL, of phenol. In one embodiment, the pharmaceutical composition comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 7.0, 7.5, 8.0, 8.5, 9.0, 9.5, or 10.0 mg/mL, of phenol.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.1 mg/mL to 10 mg/mL, preferably from 0.75 mg/mL to 1.5 mg/mL, more preferably about 1.14 mg/mL, of disodium phosphate,
from 10 mg/mL to 50 mg/mL, preferably from 20 mg/mL to 40 mg/mL, more preferably about 30 mg/mL, of glucose, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.1 mg/mL to 10 mg/mL, preferably from 0.5 mg/mL to 1 mg/mL, more preferably about 0.97 mg/mL, of tromethamine,
from 10 mg/mL to 50 mg/mL, preferably from 20 mg/mL to 40 mg/mL, more preferably about 30 mg/mL, of glucose, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.1 mg/mL to 10 mg/mL, preferably from 0.75 mg/mL to 1.5 mg/mL, more preferably about 1.14 mg/mL, of disodium phosphate,
from 20 mg/mL to 100 mg/mL, preferably from 40 mg/mL to 80 mg/mL, more preferably about 60 mg/mL, of PEG400, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.1 mg/mL to 10 mg/mL, preferably from 0.5 mg/mL to 1 mg/mL, more preferably about 0.97 mg/mL, of tromethamine,
from 20 mg/mL to 100 mg/mL, preferably from 40 mg/mL to 80 mg/mL, more preferably about 60 mg/mL, of PEG400, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.1 mg/mL to 10 mg/mL, preferably from 0.75 mg/mL to 1.5 mg/mL, more preferably about 1.14 mg/mL, of disodium phosphate,
from 5 mg/mL to 50 mg/mL, preferably from 10 mg/mL to 25 mg/mL, more preferably about 17 mg/mL, of glycerol, and
water for injection.

Advantageously, the pharmaceutical composition comprises or consists of:
from 2 mg/mL to 20 mg/mL, preferably from 4 mg/mL to 8 mg/mL, more preferably about 6 mg/mL of liraglutide,
from 0.1 mg/mL to 10 mg/mL, preferably from 0.5 mg/mL to 1 mg/mL, more preferably about 0.97 mg/mL, of tromethamine,
from 5 mg/mL to 50 mg/mL, preferably from 10 mg/mL to 25 mg/mL, more preferably about 18 mg/mL, of glycerol, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.05 mg/mL to 10 mg/mL, preferably from 0.25 mg/mL to 2.0 mg/mL more preferably about 0.47 mg/mL, of disodium phosphate dihydrate,
from 1 mg/mL to 20 mg/mL, preferably from 4 mg/mL to 6 mg/mL, more preferably about 4.7 mg/mL, of propylene glycol, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.05 mg/mL to 10 mg/mL, preferably from 0.25 mg/mL to 2.0 mg/mL, more preferably about 0.47 mg/mL, of disodium phosphate dihydrate,
from 1 mg/mL to 20 mg/mL, preferably from 4 mg/mL to 6 mg/mL, more preferably about 4.7 mg/mL, of propylene glycol,
from 0.1 mg/mL to 10 mg/mL, preferably from 1 mg/mL to 2.5 mg/mL, more preferably about 1.8 mg/mL, of phenol, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.05 mg/mL to 10 mg/mL, preferably from 0.25 mg/mL to 2.0 mg/mL, more preferably about 1.42 mg/mL, of disodium phosphate dihydrate,
from 1 mg/mL to 20 mg/mL, preferably from 12 mg/mL to 16 mg/mL, more preferably about 14 mg/mL, of propylene glycol, and
water for injection.

Advantageously, the pharmaceutical composition comprises or consists of:
from 2 mg/mL to 20 mg/mL, preferably from 4 mg/mL to 8 mg/mL, more preferably about 6 mg/mL, of liraglutide,
from 0.05 mg/mL to 10 mg/mL, preferably from 0.25 mg/mL to 2.0 mg/mL, more preferably about 1.42 mg/mL, of disodium phosphate dihydrate,
from 1 mg/mL to 20 mg/mL, preferably from 12 mg/mL to 16 mg/mL, of propylene glycol,
from 0.1 mg/mL to 10 mg/mL, preferably from 2.5 mg/mL to 7.5 mg/mL, more preferably about 5.5 mg/mL, of phenol, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.1 mg/mL to 10 mg/mL, preferably from 0.75 mg/mL to 1.5 mg/mL, more preferably about 1.14 mg/mL, of disodium phosphate,
from 10 mg/mL to 50 mg/mL, preferably from 20 mg/mL to 40 mg/mL, more preferably about 30 mg/mL, of glucose, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.1 mg/mL to 10 mg/mL, preferably from 0.5 mg/mL to 1 mg/mL, more preferably about 0.97 mg/mL, of tromethamine,
from 10 mg/mL to 50 mg/mL, preferably from 20 mg/mL to 40 mg/mL, more preferably about 30 mg/mL, of glucose, and
water for injection.

Advantageously, the pharmaceutical composition comprises or consists of:
from 0.01 mg/mL to 20 mg/mL, preferably from 0.5 mg/mL to 2 mg/mL, more preferably from 1 mg/mL to 1.5 mg/mL, even more preferably about 1.34 mg/mL, of semaglutide,
from 0.1 mg/mL to 10 mg/mL, preferably from 0.75 mg/mL to 1.5 mg/mL, more preferably about 1.14 mg/mL, of disodium phosphate,
from 20 mg/mL to 100 mg/mL, preferably from 40 mg/mL to 80 mg/mL, more preferably about 60 mg/mL, of PEG400, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.1 mg/mL to 10 mg/mL, preferably from 0.5 mg/mL to 1 mg/mL, more preferably about 0.97 mg/mL, of tromethamine,
from 20 mg/mL to 100 mg/mL, preferably from 40 mg/mL to 80 mg/mL, more preferably about 60 mg/mL, of PEG400, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.1 mg/mL to 10 mg/mL, preferably from 0.75 mg/mL to 1.5 mg/mL, more preferably about 1.14 mg/mL, of disodium phosphate,
from 5 mg/mL to 50 mg/mL, preferably from 10 mg/mL to 25 mg/mL, more preferably about 17 mg/mL, of glycerol, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.1 mg/mL to 10 mg/mL, preferably from 0.5 mg/mL to 1 mg/mL, more preferably about 0.97 mg/mL, of tromethamine,
from 5 mg/mL to 50 mg/mL, preferably from 10 mg/mL to 25 mg/mL, more preferably about 18 mg/mL, of glycerol, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.05 mg/mL to 10 mg/mL, preferably from 0.25 mg/mL to 2.0 mg/mL more preferably about 0.47 mg/mL, of disodium phosphate dihydrate,
from 1 mg/mL to 20 mg/mL, preferably from 4 mg/mL to 6 mg/mL, more preferably about 4.7 mg/mL, of propylene glycol, and
water for injection.

Advantageously, the pharmaceutical composition comprises or consists of:
from 0.01 mg/mL to 20 mg/mL, preferably from 0.5 mg/mL to 2 mg/mL, more preferably from 1 to 1.5 mg/mL, even more preferably about 1.34 mg/mL, of semaglutide,
from 0.05 mg/mL to 10 mg/mL, preferably from 0.25 mg/mL to 2.0 mg/mL, more preferably about 0.47 mg/mL, of disodium phosphate dihydrate,
from 1 mg/mL to 20 mg/mL, preferably from 4 mg/mL to 6 mg/mL, more preferably about 4.7 mg/mL, of propylene glycol,
from 0.1 mg/mL to 10 mg/mL, preferably from 1 mg/mL to 2.5 mg/mL, more preferably about 1.8 mg/mL, of phenol, and
water for injection.

Advantageously, the pharmaceutical composition comprises or consists of:
from 0.01 mg/mL to 20 mg/mL, preferably from 0.5 mg/mL to 2 mg/mL, more preferably from 1 mg/mL to 1.5 mg/mL, even more preferably about 1.34 mg/mL, of semaglutide,
from 0.05 mg/mL to 10 mg/mL, preferably from 0.25 mg/mL to 2.0 mg/mL, more preferably about 1.42 mg/mL, of disodium phosphate dihydrate,
from 1 mg/mL to 20 mg/mL, preferably from 12 mg/mL to 16 mg/mL, more preferably about 14 mg/mL, of propylene glycol, and
water for injection.

Advantageously, the pharmaceutical composition comprises or consists of:
from 0.01 mg/mL to 20 mg/mL, preferably from 0.5 mg/mL to 2 mg/mL, more preferably from 1 mg/mL to 1.5 mg/mL, even more preferably about 1.34 mg/mL, of semaglutide,
from 0.05 mg/mL to 10 mg/mL, preferably from 0.25 mg/mL to 2.0 mg/mL, more preferably about 1.42 mg/mL, of disodium phosphate dihydrate,
from 1 mg/mL to 20 mg/mL, preferably from 12 mg/mL to 16 mg/mL, of propylene glycol,
from 0.1 mg/mL to 10 mg/mL, preferably from 2.5 mg/mL to 7.5 mg/mL, more preferably about 5.5 mg/mL, of phenol, and
water for injection.

The inventors have surprisingly found that the pharmaceutical compositions according to the invention as described above induce a long-term effect when administered via intraarticular injection, in particular an effect that lasts at least three weeks, more particularly an effect that lasts at least four weeks.

### Pharmaceutical Composition for Use for Treating at Least One Joint Disease

The present invention also relates to a pharmaceutical composition according to the invention as described above for use in the treatment of at least one joint disease.

The present invention also relates to a method of treating at least one joint disease by administering to a patient in need thereof an effective amount of a pharmaceutical composition according to the invention as described above.

The present invention also relates to the use of a pharmaceutical composition according to the invention as described above for the manufacture of a medicament for the treatment of at least one joint disease.

The present invention also relates to the use of a pharmaceutical composition according to the invention as described above for the treatment of at least one joint disease.

The present invention also relates to a pharmaceutical composition according to the invention as described above for use in a method for treating at least one joint disease.

All the features described above related to the pharmaceutical composition according to the invention apply mutatis mutandis for the pharmaceutical composition according to the invention for use in the treatment of at least one joint disease, for the method of treating at least one joint disease by administering to a patient in need thereof an effective amount of a pharmaceutical composition according to the invention, for the use of a pharmaceutical composition according to the invention for the manufacture of a medicament for the treatment of at least one joint disease, for the use of a pharmaceutical composition according to the invention for the treatment of at least one joint disease and for pharmaceutical composition according to the invention for use in a method for treating at least one joint disease.

### Joint Diseases

Advantageously, said at least one joint disease is a chronic disease. The chronic disease may be osteoarthritis.

Advantageously, said at least one joint disease is an acute disease. The acute disease may be an acute pain.

Advantageously, said at least one joint disease is selected from the group consisting of osteoarthritis, joint pain and their combination. In a preferred embodiment, said at least one joint disease is a joint pain. The joint pain may be inflammatory joint pain or non-inflammatory joint pain.

The joint disease to be treated, in particular the osteoarthritis, may affect any joint, for example the joints of the hip (coxarthrosis), of the knee (gonarthrosis), of the ankle, of the foot, of the hand, of the wrist, of the elbow, of the shoulder, of the spine, and/or the temporomandibular joint. Preferably, the joint disease to be treated, in particular the osteoarthritis, affects the joints of the hip, of the knee, of the hand and/or of the rachis.

Treating at least one joint disease may comprise reducing an existing joint inflammation in a subject in need thereof.

Treating at least one joint disease may comprise increasing the chondrocyte proliferation and/or chondrocyte differentiation and/or decreasing synovitis in a subject in need thereof.

### Administration

In one embodiment, the pharmaceutical composition is administered or is to be administered via intraarticular injection, in particular via intraarticular injection into the joint cavity.

The pharmaceutical composition may be administered in combination with at least one other locally acting substances such as hyaluronic acid, nonsteroidal anti-inflammatory drugs, stem cells, growth factors (such as sprifermine or BMP7), MMPs inhibitors, Wnt inhibitors and/or analgesic substances.

Advantageously, the pharmaceutical composition is administered or is to be administered at a dose from 0.7 μg to 180 μg of liraglutide, preferably from 20 μg to 180 μg, more preferably from 20 μg to 120 μg, of liraglutide. One dose refers to the cumulative amount of GLP-1R agonists administered in 2 weeks to 1 month. In one embodiment, the pharmaceutical composition is administered or is to be administered at a dose of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 μg, of liraglutide.

Advantageously, the pharmaceutical composition is administered or is to be administered at a dose from 0.7 μg to 180 μg of liraglutide, preferably from 20 μg to 180 μg, more preferably from 20 μg to 120 μg, of liraglutide. One dose refers to the cumulative amount of GLP-1R agonists administered in 2 weeks to 1 month. In one embodiment, the pharmaceutical composition is administered or is to be administered at a dose of 0.7, 1, 2, 2.2, 3, 4, 5, 6, 6.7, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 μg, of liraglutide.

Advantageously, the pharmaceutical composition is administered or is to be administered at a dose from 0.7 μg to 180 μg of semaglutide, preferably from 20 μg to 180 μg, more preferably from 20 μg to 120 μg, of semaglutide. One dose refers to the cumulative amount of GLP-1R agonists administered in 2 weeks to 1 month. In one embodiment, the pharmaceutical composition is administered or is to be administered at a dose of 0.7, 1, 2, 2.2, 3, 4, 5, 6, 6.7, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 μg, of semaglutide.

Advantageously, the pharmaceutical composition is administered or is to be administered at a dose from 0.0245 mg to 6.3 mg of liraglutide, preferably from 0.7 mg to 6.3 mg, of liraglutide. According to an embodiment, the pharmaceutical composition is administered or is to be administered at a dose from 0.0245 mg to 6.3 mg of liraglutide, preferably from 0.3 mg to 6.0 mg of liraglutide. One dose refers to the cumulative amount of GLP-1R agonists administered in 2 weeks to 1 month. In one embodiment, the pharmaceutical composition is administered or is to be administered at a dose of 0.0245, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00, 6.05, 6.10, 6.15, 6.20, 6.25 or 6.3 mg, of liraglutide. In one embodiment, the pharmaceutical composition is administered or is to be administered at a dose of 0.3 mg, 1.0 mg, 3.0 mg or 6.0 mg, of liraglutide.

Advantageously, the pharmaceutical composition is administered or is to be administered at a dose from 0.0245 mg to 6.3 mg of semaglutide, preferably from 0.7 mg to 6.3 mg, preferably from 0.25 to 1 mg of semaglutide. One dose refers to the cumulative amount of GLP-1R agonists administered in 2 weeks to 1 month. In one embodiment, the pharmaceutical composition is administered or is to be administered at a dose of 0.0245, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00, 6.05, 6.10, 6.15, 6.20, 6.25 or 6.3 mg, of semaglutide. In one embodiment, the pharmaceutical composition is administered or is to be administered at a dose of 0.25, 0.5 or 1 mg, of semaglutide.

Advantageously, said dose of said pharmaceutical composition is administered or is to be administered in one or at least two intraarticular injections.

Advantageously, said dose of said pharmaceutical composition is administered or is to be administered in one or at least two intraarticular injections in the same joint.

In one embodiment, said dose of said pharmaceutical composition is administered or is to be administered in one intraarticular injection. For example, the dose is 20 μg of liraglutide or semaglutide and is administered or is to be administered in one intraarticular injection. For example, the dose is 20 μg of liraglutide and is administered or is to be administered in one intraarticular injection. For example, the dose is 20 μg of semaglutide and is administered or is to be administered in one intraarticular injection. For another example, the dose is 0.3 mg, 1.0 mg, 3.0 mg or 6.0 mg of liraglutide and is administered or is to be administered in one intraarticular injection. For another example, the dose is 0.25, 0.5 or 1 mg of semaglutide and is administered or is to be administered in one intraarticular injection. This intraarticular injection may be repeated every two weeks. This intraarticular injection may be repeated every three weeks. This intraarticular injection may be repeated every month.

Advantageously, the dose is 0.3 mg of liraglutide and is administered or is to be administered in one intraarticular injection, the intraarticular injection being repeated every two weeks, every three weeks or every month.

Advantageously, the dose is 1.0 mg of liraglutide and is administered or is to be administered in one intraarticular injection, the intraarticular injection being repeated every two weeks, every three weeks or every month.

Advantageously, the dose is 3.0 mg of liraglutide and is administered or is to be administered in one intraarticular injection, the intraarticular injection being repeated every two weeks, every three weeks or every month.

Advantageously, the dose is 6.0 mg of liraglutide and is administered or is to be administered in one intraarticular injection, the intraarticular injection being repeated every two weeks, every three weeks or every month.

Advantageously, the dose is 0.25 mg of semaglutide and is administered or is to be administered in one intraarticular injection, the intraarticular injection being repeated every two weeks, every three weeks or every month.

Advantageously, the dose is 0.5 mg of semaglutide and is administered or is to be administered in one intraarticular injection, the intraarticular injection being repeated every two weeks, every three weeks or every month.

Advantageously, the dose is 1 mg of semaglutide and is administered or is to be administered in one intraarticular injection, the intraarticular injection being repeated every two weeks, every three weeks or every month.

In another embodiment, said dose of said pharmaceutical composition is administered or is to be administered in two intraarticular injections, for example both intraarticular injections are administered or are to be administered on the same day or both injections are administered or are to be administered on different days, notably in the same joint. In particular, the dose of the pharmaceutical composition may be administered in two intraarticular injections: for example, the first at J1 and the second at J8 or the first at J1 and the second at J15.

When the dose is administered or is to be administered in multiple injections, each injection may contain the same amount of GLP-1R agonist or varying amounts of GLP-1R agonist. For example, the dose is 20 μg of liraglutide and is administered or is to be administered in two intraarticular injections of 10 μg of liraglutide: the first injection at J1 and the second injection at J8, or the first injection at J1 and the second injection at J15; these two injections may be repeated every three weeks, that is that the further first injection is at J22. These two injections may also be repeated every month, that is that the further first injection is at J29. In the case were the first injection is at J1 and the second injection is at J8, two injections may also be repeated every two weeks, that is that the further first injection is at J15. For example, the dose is 0.3 mg, 1.0 mg, 3.0 mg or 6.0 mg of liraglutide and is administered or is to be administered in two intraarticular injections of respectively 0.15 mg, 0.5 mg, 1.5 mg or 3.0 mg of liraglutide: the first injection at J1 and the second injection at J8, or the first injection at J1 and the second injection at J15; these two injections may be repeated every three weeks, that is that the further first injection is at J22. These two injections may also be repeated every month, that is that the further first injection is at J29. In the case were the first injection is at J1 and the second injection is at J8, two injections may also be repeated every two weeks, that is that the further first injection is at J15. For still another example, the dose is 20 μg of semaglutide and is administered or is to be administered in two intraarticular injections of 10 μg of semaglutide: the first injection at J1 and the second injection at J8, or the first injection at J1 and the second injection at J15; these two injections may be repeated every three weeks, that is that the further first injection is at J22. These two injections may also be repeated every month, that is that the further first injection is at J29. In the case were the first injection is at J1 and the second injection is at J8, two injections may also be repeated every two weeks, that is that the further first injection is at J15. For still another example, the dose is 0.25, 0.5 or 1 mg of semaglutide and is administered or is to be administered in two intraarticular injections of respectively 0.125 mg, 0.25 mg or 0.5 mg of semaglutide: the first injection at J1 and the second injection at J8, or the first injection at J1 and the second injection at J15; these two injections may be repeated every three weeks, that is that the further first injection is at J22. These two injections may also be repeated every month, that is that the further first injection is at J29. In the case were the first injection is at J1 and the second injection is at J8, two injections may also be repeated every two weeks, that is that the further first injection is at J15.

Indeed, the inventors of the present invention have surprisingly discovered that two intraarticular injections of a half-dose of liraglutide (one injection at J1 and one injection at J8) induce the same analgesic effect and the same duration of analgesic effect as one intraarticular injection of the dose of liraglutide (at J1). For example, two intraarticular injections of 10 μg of liraglutide (one injection at J1 and one injection at J8) induce the same analgesic effect and the same duration of analgesic effect as one intraarticular injection of 20 μg of liraglutide (at J1).

In one embodiment, several doses of the pharmaceutical composition are administered or are to be administered to the subject, the doses being administered every two weeks to every month. In one preferred embodiment, several doses of the pharmaceutical composition are administered or are to be administered to the subject, the doses being administered every three weeks. In one more preferred embodiment, several doses of the pharmaceutical composition are administered or are to be administered to the subject, the doses being administered every month.

Indeed, the inventors of the present invention have surprisingly discovered that the pharmaceutical compositions according to the invention have a three weeks long-lasting analgesic effect following acute intraarticular administration, in particular a four weeks long-lasting analgesic effect following acute intraarticular administration.

### BRIEF DESCRIPTION OF THE DRAWINGS

FIG. 1: IC₅₀ determination on NO secretion by different concentrations of Liraglutide pre-formulation 1 ranging from 4.1 nM to 3 μM in LPS-stimulated conditions (nM). Mean±SEM, n=4 (each condition performed in quadruplicates)

FIG. 2: IC₅₀ determination on NO secretion by different concentrations of Liraglutide pre-formulation 2 ranging from 4.1 nM to 3 μM in LPS-stimulated conditions (nM). Mean±SEM, n=4 (each condition performed in quadruplicates).

FIG. 3: IC₅₀ determination on NO secretion by different concentrations of Liraglutide pre-formulation 3 ranging from 4.1 nM to 3 μM in LPS-stimulated conditions (nM). Mean±SEM, n=4 (each condition performed in quadruplicates)

FIG. 4: IC₅₀ determination on NO secretion by different concentrations of Liraglutide pre-formulation 4 ranging from 4.1 nM to 3 μM in LPS-stimulated conditions (nM). Mean±SEM, n=4 (each condition performed in quadruplicates)

FIG. 5: IC₅₀ determination on NO secretion by different concentrations of Liraglutide pre-formulation 5 ranging from 4.1 nM to 3 μM in LPS-stimulated conditions (nM). Mean±SEM, n=4 (each condition performed in quadruplicates)

FIG. 6: IC₅₀ determination on NO secretion by different concentrations of Liraglutide pre-formulation 6 ranging from 4.1 nM to 3 μM in LPS-stimulated conditions (nM). Mean±SEM, n=4 (each condition performed in quadruplicates)

FIG. 7: IC₅₀ determination on NO secretion by different concentrations of Liraglutide pre-formulation 7 ranging from 4.1 nM to 3 μM in LPS-stimulated conditions (nM). Mean±SEM, n=4 (each condition performed in quadruplicates)

FIG. 8: IC₅₀ determination on NO secretion by different concentrations of Liraglutide pre-formulation 8 ranging from 4.1 nM to 3 μM in LPS-stimulated conditions (nM). Mean±SEM, n=4 (each condition performed in quadruplicates)

FIG. 9: IC₅₀ determination on NO secretion by different concentrations of Liraglutide pre-formulation 9 ranging from 4.1 nM to 3 μM in LPS-stimulated conditions (nM). Mean±SEM, n=4 (each condition performed in quadruplicates)

FIG. 10: IC₅₀ determination on NO secretion by different concentrations of Liraglutide pre-formulation 10 ranging from 4.1 nM to 3 μM in LPS-stimulated conditions (nM). Mean±SEM, n=4 (each condition performed in quadruplicates)

FIG. 11: IC₅₀ determination on NO secretion by different concentrations of Victoza® ranging from 4.1 nM to 3 μM in LPS-stimulated conditions (nM). Mean±SEM, n=4 (each condition performed in quadruplicates)

FIG. 12: IC₅₀ determination on NO secretion by different concentrations of Liraglutide pre-formulation 2 ranging from 4.1 nM to 3 μM in LPS-stimulated conditions (nM). Mean±SEM, n=4 (each condition performed in quadruplicates)

FIG. 13: IC₅₀ determination on NO secretion by different concentrations of Liraglutide pre-formulation 6 ranging from 4.1 nM to 3 μM in LPS-stimulated conditions (nM). Mean±SEM, n=4 (each condition performed in quadruplicates)

FIG. 14: IC₅₀ determination on NO secretion by different concentrations of Victoza® (PBS) ranging from 4.1 nM to 3 μM in IL-10-stimulated conditions (nM). Mean±SEM, n=4 (each condition performed in quadruplicates)

FIG. 15: Von Frey test withdrawal threshold on right hind paw up to day 11. Mean±SEM, n=8 per group. FIG. 15A: Preformulation-2 compared to control vehicle, MIA/vehicle and Victoza (20 μg) group. FIG. 15B: Preformulation-6 compared to control vehicle, MIA/vehicle and Victoza (20 μg) group.
*** p<0.001 Control/Vehicle (1M) vs MIA/vehicle group (2M) at D2, D7 and D10;
*** p<0.001 Victoza 20 μg group (3M) vs MIA/vehicle group (2M) at D7 and D10;
*** p<0.001 MIA/vehicle group (2M) vs Preformulation-2 and -6 at 20 μg and 120 μg group (9M, 8M, 6M and 5M) at D7 and D10;
** p<0.001 MIA/vehicle group (2M) vs Preformulation-2 at 3.3 μg group (4M) at D7;
*** p<0.001 MIA/vehicle group (2M) vs Preformulation-2 at 3.3 μg group (4M) at D10;
** p<0.01 MIA/vehicle group (2M) vs Preformulation-2 at 3.3 μg group (7M) at D7; and
** p<0.01 MIA/vehicle group (2M) vs Preformulation-2 at 3.3 μg group (7M) at D10.

FIG. 16: Von Frey test withdrawal threshold on right hind paw up to day 32. Mean±SEM, n=8 per group. ** p<0.01, *** p<0.001 vs MIA/vehicle group 2M.

FIG. 17: von Frey test withdrawal threshold on right hind paw up to day 31. Mean±SEM, n=8 per group. FIG. 17A: three of the six doses of Preformulation-2 compared to control vehicle, MIA/vehicle, MIA/Victoza® (20 μg) and MIA/Ozempic® (20 μg)-groups. FIG. 17B: the three other doses of Preformulation-2 compared to control vehicle, MIA/vehicle, MIA/Victoza® (20 μg) and MIA/Ozempic® (20 μg)-groups.
*** p<0.001 Control/Vehicle (11M) vs MIA/vehicle group (12M) at D2, D7, D10, D18, D25 and D31;
** p<0.01 Preformulation-2 0.7 μg group (13M) vs MIA/vehicle group (2M) at D7;
*p<0.05 Preformulation-2 0.7 μg group (13M) vs MIA/vehicle group (2M) at D10;
** p<0.01 Preformulation-2 2.2 μg group (14M) vs MIA/vehicle group (2M) at D7 and D10;
*** p<0.001 Preformulation-2 6.7 μg, 20 μg and 60 μg group (15M, 16M and 17M)
vs MIA/vehicle group (2M) at D7 and D10;
*** p<0.001 Preformulation-2 180 μg group (18M) vs MIA/vehicle group (2M) at D7, D10, D18, D25 and D31;
*** p<0.001 Victoza 20 μg group (19M) vs MIA/vehicle group (2M) at D7, D10, D18, D25 and D31;
*** p<0.001 Ozempic 20 μg group (19M) vs MIA/vehicle group (2M) at D7, D10, D18, D25 and D31.

FIG. 18: Von Frey filament test withdrawal threshold results on right hind paw. ^{$$$} p<0.001 Control/vehicle group (31M) vs MIA/vehicle group and treated groups (32M, 33M, 34M, 35M, 36M and 37M) at day 2.
*p<0.05 MIA/vehicle group (32M) vs exenatide (E2)-treated group (35M) at day 7.
** p<0.01 MIA/vehicle group (32M) vs lixisenatide-treated group (36M) at day 7.
** p<0.01 MIA/vehicle group (32M) vs exenatide (E1)-treated group (34M) at day 10 and 30.
*** p<0.001 MIA/vehicle group (32M) vs liraglutide-treated group (37M) at day 7, 10, 18, 24 and 30.
*** p<0.001 MIA/vehicle group (32M) vs lixisenatide-treated group (36M) at day 10, 18, 24 and 30.
*** p<0.001 MIA/vehicle group (32M) vs exenatide (E2)-treated group (35M) at day 10, 18, 24 and 30.
*** p<0.001 MIA/vehicle group (32M) vs exenatide (E1)-treated group (34M) at day 10, 18 and 24.
*** p<0.001 MIA/vehicle group (32M) vs dulaglutide-treated group (33M) at day 10, 18, 24 and 30.
*** p<0.001 MIA/vehicle group (32M) vs Vehicle/control group (31M) at day 2, 7, 10, 18, 24 and 30. Until day 7, mean±SEM, n=4 per group. From day 10, mean±SEM, n=7-8 per group. 31M-Vehicle group scores were all 15 (g) or plus.

FIG. 19: Mechanical pain evaluation (incapacitance test) for Groups 41M, 42M, 43M and 44M. *p<0.05, **p<0.01 and ***p<0.001. There was no significant difference between the control group 41M and the treated groups 43M and 44M so they are not represented on FIG. 19.

FIG. 20: Von Frey test withdrawal threshold results on right hind paw. MIA/Vehicle (46M) vs Control/Vehicle (45M) (Mann-Whitney test; *** p<0,001). MIA/Vehicle (46M) vs MIA preformulation-2 and pre-formulation 11 (47M and 48M) (Mann-Whitney test; * p<0.05, ** p<0.01, *** p<0,001).

FIG. 21: Plasma dosage of liraglutide for groups 51M, 52M and 53M.

FIG. 22: Synovial fluid dosage of liraglutide for groups 54M, 55M and 56M.

### EXAMPLES

The present invention is further illustrated by the following examples.

### Example 1: pH and Osmolality of Compositions According to the Present Invention

Six pharmaceutical compositions were prepared.

Pre-formulation 1 contained: 6 mg/mL of liraglutide, 8 mM of disodium phosphate, 30 mg/mL of glucose and water for injection.

Pre-formulation 2 contained: 6 mg/mL of liraglutide, 8 mM of tromethamine, 30 mg/mL of glucose and water for injection.

Pre-formulation 5 contained: 6 mg/mL of liraglutide, 8 mM of disodium phosphate, 60 mg/mL of PEG400 and water for injection.

Pre-formulation 6 contained: 6 mg/mL of liraglutide, 8 mM of tromethamine, 60 mg/mL of PEG400 and water for injection.

Pre-formulation 7 contained: 6 mg/mL of liraglutide, 8 mM of disodium phosphate, 17 mg/mL of glycerol and water for injection.

Pre-formulation 8 contained: 6 mg/mL of liraglutide, 8 mM of tromethamine, 18 mg/mL of glycerol and water for injection.

The pH and the osmolality of the compositions were measured:

TABLE 1

| | | Osmolality (mmol/kg) | | |
|---|---|---|---|---|
| | pH | T0 | T0 + 48 hours | T0 + 1 month |
| Preformulation 1 | 8.2 | 207 | 207 | 210 |
| Preformulation 2 | 8.6 | 209 | 211 | 215 |
| Preformulation 5 | 8.4 | 202 | 210 | 220 |
| Preformulation 6 | 8.6 | 198 | 203 | 216 |
| Preformulation 7 | 8.4 | 202 | 206 | 212 |
| Preformulation 8 | 8.6 | 181 | 189 | 199 |

The results of osmolality and particle size measurements, are presented in the following table:

TABLE 2

| Pre- | | | Osmolality (mmol/kg) | | | Particles size (nm) | | |
|---|---|---|---|---|---|---|---|---|
| formulation | Composition | pH | T0 | T + 48 h | T + 1 Mo | T0 | T + 48 h | T + 1 Mo |
| 1 | Glucose + | 8.2 | 207 | 207 | 210 | 110 | 70 | 234 |
| | Phosphate | | | | | | | |
| 2 | Glucose + | 8.6 | 209 | 211 | 215 | 313 | 227 | 100 |
| | Tromethamine | | | | | | | |
| 5 | PEG400 + | 8.4 | 202 | 210 | 220 | 129 | 37 | 110 |
| | Phosphate | | | | | | | |
| 6 | PEG400 + | 8.6 | 198 | 203 | 216 | 329 | 148 | 347 |
| | Tromethamine | | | | | | | |
| 7 | Glycerol + | 8.4 | 202 | 206 | 212 | 68 | 46 | 70 |
| | Phosphate | | | | | | | |
| 8 | Glycerol + | 8.6 | 181 | 189 | 199 | 412 | 239 | 400 |
| | Tromethamine | | | | | | | |

It appeared that the six compositions had a pH allowing the solubilization of liraglutide. Moreover, the osmolality values after 48 hours and after one month were similar to the ones obtained at TO. The compositions osmolality was therefore stable.

### Example 2: In Vitro Efficacy of 10 Compositions According to the Invention on Murine Macrophages Cell Line RAW 264.7

### Test System

The cell line used is RAW 264.7 (Macrophages from Balb/C male mice transformed by Abelson murine leukemia virus), which ATCC reference is TIB-71.

### Test Items and Test Items Vehicle

TABLE 3

| | | | Liraglutide |
|---|---|---|---|
| Pre-formulation | | | API |
| name | Isotonic agent | Buffer agent | (6 mg/ml) |
| Pre-formulation | Glucose | Disodium phosphate (Na₂HPO₄) | yes |
| 1 | | (8 mM) | |
| Pre-formulation | Glucose | Tromethamine | yes |
| 2 | | | |
| Pre-formulation | Propylene | Tromethamine | yes |
| 3 | glycol | | |
| Pre-formulation | — | Disodium phosphate and | yes |
| 4 | | monosodium phosphate | |
| | | (Na₂HPO₄/NaH₂PO₄) (VWR | |
| | | 28020.292-18D044123) (VWR | |
| | | 28011.260-18C224139 | |
| | | (≈130 mM, iv DRUGABILIS | |
| | | buffer) | |
| Pre-formulation | PEG400 | Disodium phosphate (Na₂HPO₄) | yes |
| 5 | | (8 mM) | |
| Pre-formulation | PEG400 | Tromethamine | yes |
| 6 | | | |
| Pre-formulation | Glycerol | Disodium phosphate (Na₂HPO₄) | yes |
| 7 | | (8 mM) | |
| Pre-formulation | Glycerol | Tromethamine | yes |
| 8 | | | |
| Pre-formulation | — | Dulbecco's Phosphate Buffered | yes |
| 9 | | Saline | |
| Pre-formulation | Propylene | Disodium phosphate (Na₂HPO₄) | yes |
| 10 | glycol | (8 mM) | |

Pre-formulations 1, 2, 5, 6, 7 and 8 of example 2 are identical to pre-formulations respectively 1, 2, 5, 6, 7 and 8 of example 1.

### Formulation

Except when another reference is indicated, for the ten pre-formulations, the glucose is Sigma 16325-SZBF2860V, the disodium phosphate is Sigma-04276-90900, the tromethamine is Sigma T6687-WXBC2569V, the Dulbecco's Phosphate Buffered Saline is Sigma D8662-RNBJ0600, the propylene glycol is Sigma 16033-SZBC2900V, the PEG400 is Sigma 81172-BCBT2825, and the glycerol is Fluka 49783-BCBD0423.

LPS (Sigma) supplied as a powder was prepared at 1 mg/ml in PBS. The concentrated stock solution was diluted to the final concentration of 100 ng/ml in DMEM treatment medium for experiment.

Pre-formulations of Liraglutide (1 to 10) supplied as solutions at 6 mg/ml of liraglutide were diluted in PBS as a stock solution. The concentrated stock solution was then diluted in DMEM treatment medium to reach the final concentrations of 3 μM, 1 μM, 333.3 nM, 111.1 nM, 37.0 nM, 12.3 nM and 4.1 nM.

Victoza® (Novo nordisk) is a commercial Liraglutide drug. Test item supplied as a solution at 6 mg/ml was diluted in PBS as a stock solution. The concentrated stock solution was then diluted in DMEM treatment medium to reach the final concentrations of 3 μM, 1 μM, 333.3 nM, 111.1 nM, 37.0 nM, 12.3 nM and 4.1 nM. Victoza® and the test items obtained from Victoza® are compositions according to the invention, as they comprise a GLP-1R agonist (liraglutide), a phosphate buffer and propylene glycol as isotonic agent.

The vehicle (PBS) is supplied “ready to use” and was diluted in cell treatment medium at a final concentration of 1:100.

### Experimental Design

### Cell Culture

The cells were cultured until 80% confluence and harvested using a cell scraper and resuspended in a new flask; until enough cells are obtained to start the study.

### Seeding

The cells were harvested at 70-80% confluence using a cell scraper, counted and re-suspended to a final concentration of 1×10⁶ cells/ml in seeding medium (DMEM High glucose+1% P/S). Next, the cells were seeded in 96-well microtiter plate (100 μl/well −100 000 cells/well). The cell cultures were maintained under sterile condition in an incubator for 24 h at 37° C. with 5% CO₂.

### Treatment

The seeding medium was aspirated from the 96-microtiter plate. Then, 200 μl of medium containing vehicle or LPS at 100 ng/ml with different doses of pre-formulated Liraglutide or Victoza® (4.1 nM to 3 μM) or vehicles were added to each well according to Table 4 for study design and Table 5 for study timeline. The plates were incubated 24 hours at 37° C. with 5% CO₂.

TABLE 4
Study design

| Group | Treatment | Induction | Time |
|---|---|---|---|
| 1 | Cells only (Blank) | Vehicle (PBS) | 24 hours |
| 2 | Vehicle (PBS) | LPS 100 ng/ml | co-treatment |
| 3 | Pre-formulation 1 4.1 nM | | |
| 4 | Pre-formulation 1 12.3 nM | | |
| 5 | Pre-formulation 1 37.0 nM | | |
| 6 | Pre-formulation 1 111.1 nM | | |
| 7 | Pre-formulation 1 333.3 nM | | |
| 8 | Pre-formulation 1 1 μM | | |
| 9 | Pre-formulation 1 3 μM | | |
| 10 | Pre-formulation 2 4.1 nM | | |
| 11 | Pre-formulation 2 12.3 nM | | |
| 12 | Pre-formulation 2 37.0 nM | | |
| 13 | Pre-formulation 2 111.1 nM | | |
| 14 | Pre-formulation 2 333.3 nM | | |
| 15 | Pre-formulation 2 1 μM | | |
| 16 | Pre-formulation 2 3 μM | | |
| 17 | Pre-formulation 3 4.1 nM | | |
| 18 | Pre-formulation 3 12.3 nM | | |
| 19 | Pre-formulation 3 37.0 nM | | |
| 20 | Pre-formulation 3 111.1 nM | | |
| 21 | Pre-formulation 3 333.3 nM | | |
| 22 | Pre-formulation 3 1 μM | | |
| 23 | Pre-formulation 3 3 μM | | |
| 24 | Pre-formulation 4 4.1 nM | | |
| 25 | Pre-formulation 4 12.3 nM | | |
| 26 | Pre-formulation 4 37.0 nM | | |
| 27 | Pre-formulation 4 111.1 nM | | |
| 28 | Pre-formulation 4 333.3 nM | | |
| 29 | Pre-formulation 4 1 μM | | |
| 30 | Pre-formulation 4 3 μM | | |
| 31 | Pre-formulation 5 4.1 nM | | |
| 32 | Pre-formulation 5 12.3 nM | | |
| 33 | Pre-formulation 5 37.0 nM | | |
| 34 | Pre-formulation 5 111.1 nM | | |
| 35 | Pre-formulation 5 333.3 nM | | |
| 36 | Pre-formulation 5 1 μM | | |
| 37 | Pre-formulation 5 3 μM | | |
| 38 | Pre-formulation 6 4.1 nM | | |
| 39 | Pre-formulation 6 12.3 nM | | |
| 40 | Pre-formulation 6 37.0 nM | | |
| 41 | Pre-formulation 6 111.1 nM | | |
| 42 | Pre-formulation 6 333.3 nM | | |
| 43 | Pre-formulation 6 1 μM | | |
| 44 | Pre-formulation 6 3 μM | | |
| 45 | Pre-formulation 7 4.1 nM | | |
| 46 | Pre-formulation 7 12.3 nM | | |
| 47 | Pre-formulation 7 37.0 nM | | |
| 48 | Pre-formulation 7 111.1 nM | | |
| 49 | Pre-formulation 7 333.3 nM | | |
| 50 | Pre-formulation 7 1 μM | | |
| 51 | Pre-formulation 7 3 μM | | |
| 52 | Pre-formulation 8 4.1 nM | | |
| 53 | Pre-formulation 8 12.3 nM | | |
| 54 | Pre-formulation 8 37.0 nM | | |
| 55 | Pre-formulation 8 111.1 nM | | |
| 56 | Pre-formulation 8 333.3 nM | | |
| 57 | Pre-formulation 8 1 μM | | |
| 58 | Pre-formulation 8 3 μM | | |
| 59 | Pre-formulation 9 4.1 nM | | |
| 60 | Pre-formulation 9 12.3 nM | | |
| 61 | Pre-formulation 9 37.0 nM | | |
| 62 | Pre-formulation 9 111.1 nM | | |
| 63 | Pre-formulation 9 333.3 nM | | |
| 64 | Pre-formulation 9 1 μM | | |
| 65 | Pre-formulation 9 3 μM | | |
| 66 | Pre-formulation 10 4.1 nM | | |
| 67 | Pre-formulation 10 12.3 nM | | |
| 68 | Pre-formulation 10 37.0 nM | | |
| 69 | Pre-formulation 10 111.1 nM | | |
| 70 | Pre-formulation 10 333.3 nM | | |
| 71 | Pre-formulation 10 1 μM | | |
| 72 | Pre-formulation 10 3 μM | | |
| 73 | Victoza ® 4.1 nM (in PBS) | | |
| 74 | Victoza ® 12.3 nM (in PBS) | | |
| 75 | Victoza ® 37.0 nM (in PBS) | | |
| 76 | Victoza ® 111.1 nM (in PBS) | | |
| 77 | Victoza ® 333.3 nM (in PBS) | | |
| 78 | Victoza ® 1 μM (in PBS) | | |
| 79 | Victoza ® 3 μM (in PBS) | | |

Each condition treatment was run in quadruplicate.

TABLE 5
Study schedule

| | Study day | | | | |
|---|---|---|---|---|---|
| | | | | | After |
| Procedure | Before study | 1 | 2 | 3 | study |
| RAW 264.7 cell culture | ✓ | | | | |
| Plating and starving | | ✓ | | | |
| RAW 264.7 cells | | | | | |
| Stimulation with LPS +/− | | | ✓ | | |
| Liraglutide or | | | | | |
| Victoza ® or vehicles | | | | | |
| Collect the supernatant | | | | ✓ | |
| Dosages NO | | | | | ✓ |

### Tests and Evaluations

At study termination, culture medium (±200 μl) of each well was collected in 1.5 ml tube (1 tube per well), centrifuged at 4000 rpm at room temperature and supernatant were added to a new 1.5 ml tube.

### Nitrite Oxide Dosage (Griess Reagent)

Nitrite reagent assay was performed according to Manufacturer's instructions (Nitrite Reagent Assay, Promega). The test system is based on a chemical reaction which transform sulfanilamide in azo compound in presence of nitrite ions (NO₂⁻) and N-1-napthylethylenediamine dihydrochloride (NED). Azo compound coloration is detectable at 540 nm. The solutions are “ready to use”. Nitrite standard was diluted in culture medium to obtain a reference curve (0-100 μM) for quantification. 50 μl of blank, standard or culture supernatant were added to wells of a 96-microtiter plate. 50 μl of Sulfanilamide Solution were added to the wells and the plate was incubated for 5-10 minutes in the dark. Then, 50 μl of NED Solution were added to the wells and the plate was incubated for 5-10 minutes in the dark. A purple/magenta color forms immediately and the absorbance was measured within 30 minutes at 540 nm. The average optical density (OD) of read blank wells was subtracted from each reading. Concentration of nitrite ions was calculated from the reference curve.

As expected, NO production in vehicle conditions was below the limit of detection and were attributed a value of 1.56 μM. In LPS-stimulated conditions, detection of nitrite oxide in culture medium is significantly increased compared to the vehicle. This secretion was significantly reduced by each tested-dose of liraglutide compositions according to the invention (i.e. by pre-formulations 1 to 10 and by Victoza® test items) compared to the LPS alone condition, with a sigmoidal dose response pattern. Results are presented in FIG. 1 to 11. IC₅₀ was calculated for each formulation using Prism software and summary results are presented in Table 6.

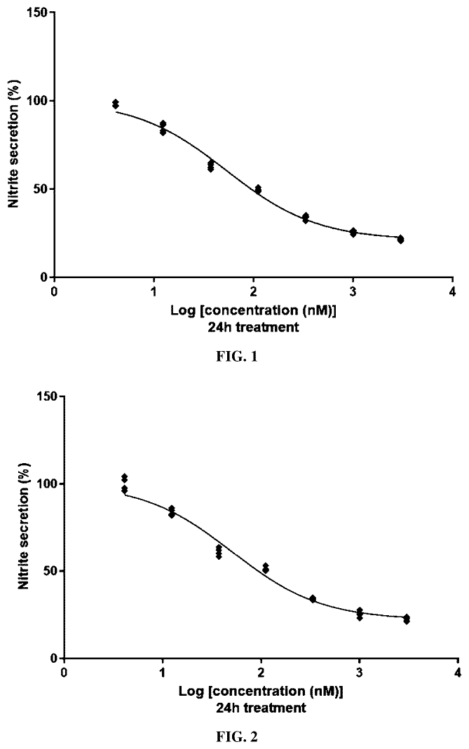

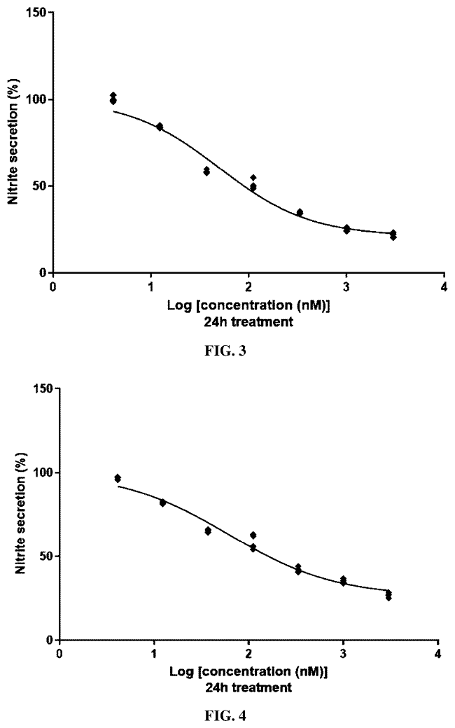

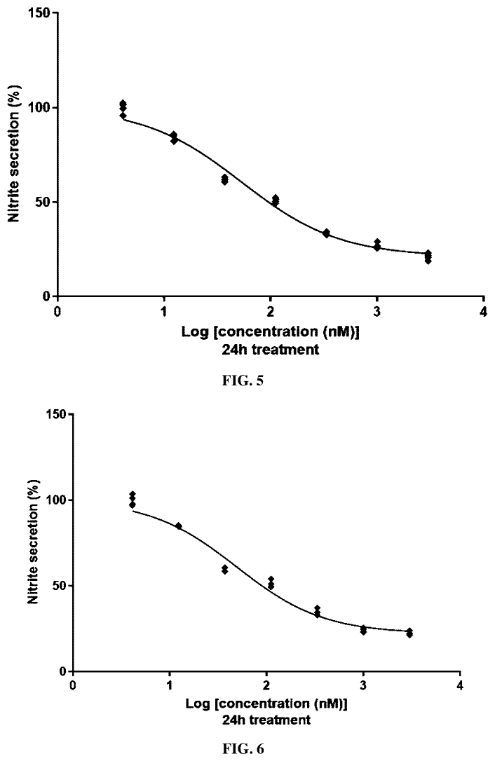

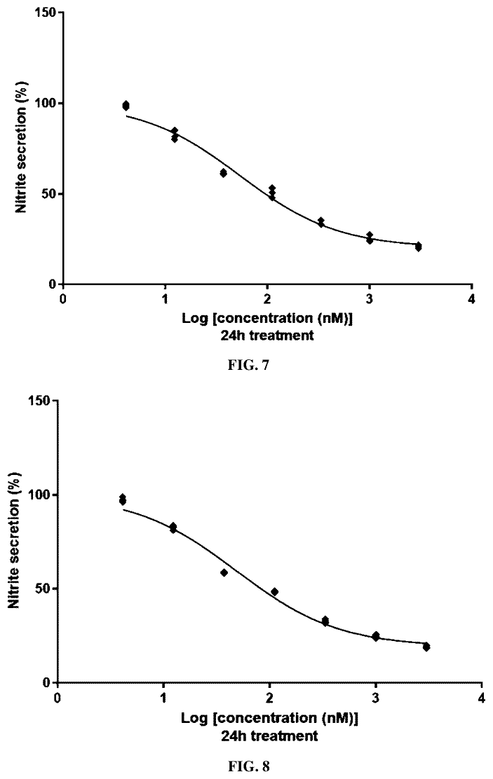

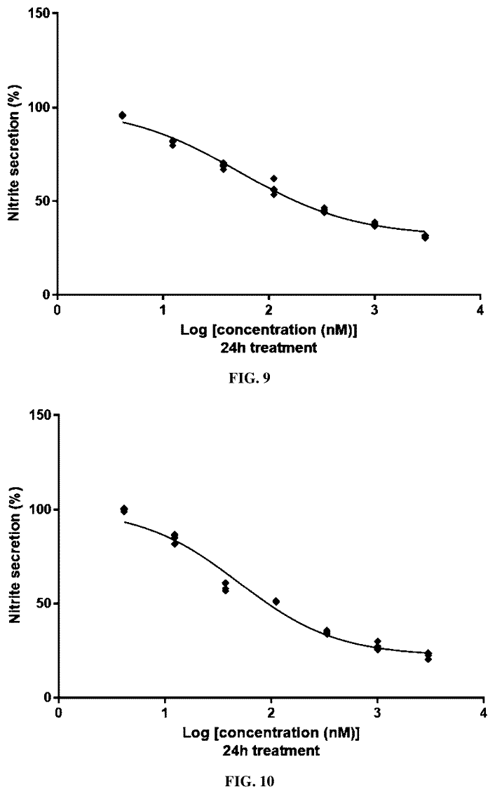

For RAW 264.7 cells cultured in high glucose conditions, Liraglutide pre-formulation 1 IC₅₀ dose on NO secretion is 53 nM with a confidence interval between 48 nM and 59 nM (FIG. 1); Liraglutide pre-formulation 2 IC₅₀ dose on NO secretion is 51 nM with a confidence interval between 44 nM and 59 nM (FIG. 2); Liraglutide pre-formulation 3 IC₅₀ dose on NO secretion is 50 nM with a confidence interval between 43 nM and 59 nM (FIG. 3); Liraglutide pre-formulation 4 IC₅₀ dose on NO secretion is 61 nM with a confidence interval between 52 nM and 72 nM (FIG. 4); Liraglutide pre-formulation 5 IC₅₀ dose on NO secretion is 53 nM with a confidence interval between 47 nM and 61 nM (FIG. 5); Liraglutide pre-formulation 6 IC₅₀ dose on NO secretion is 49 nM with a confidence interval between 42 nM and 57 nM (FIG. 6); Liraglutide pre-formulation 7 IC₅₀ dose on NO secretion is 54 nM with a confidence interval between 47 nM and 62 nM (FIG. 7); Liraglutide pre-formulation 8 IC₅₀ dose on NO secretion is 48 nM with a confidence interval between 43 nM and 55 nM (FIG. 8); Liraglutide pre-formulation 9 IC₅₀ dose on NO secretion is 53 nM with a confidence interval between 47 nM and 59 nM (FIG. 9); Liraglutide pre-formulation 10 IC₅₀ dose on NO secretion is 50 nM with a confidence interval between 42 nM and 58 nM (FIG. 10) and Victoza® (PBS) IC₅₀ dose on NO secretion is 52 nM with a confidence interval between 46 nM and 58 nM (FIG. 11).

TABLE 6
IC₅₀ summary table

| Formulation according | |
|---|---|
| to the invention | IC₅₀ value |
| Victoza ® (PBS) | 52 nM (46-58) |
| Pre-formulation 1 | 53 nM (48-59) |
| Pre-formulation 2 | 51 nM (44-59) |
| Pre-formulation 3 | 50 nM (43-59) |
| Pre-formulation 4 | 61 nM (52-72) |
| Pre-formulation 5 | 53 nM (47-61) |
| Pre-formulation 6 | 49 nM (42-57) |
| Pre-formulation 7 | 54 nM (47-62) |
| Pre-formulation 8 | 48 nM (43-55) |
| Pre-formulation 9 | 53 nM (47-59) |
| Pre-formulation 10 | 50 nM (42-58) |

### Conclusion

The objective of the study was to test 7 doses of 10 Liraglutide pre-formulations in comparison to Victoza®, a marketed Liraglutide drug (4.1 nM to 3 μM) in RAW 264.7 cells, a murine macrophage cell line.

We demonstrated that all compositions according to the invention (i.e. liraglutide pre-formulations 1 to 10 and Victoza® test items) were able to dose-dependently inhibit the LPS-induced NO production in RAW 264.7 cells.

Liraglutide pre-formulations 1 to 10, Liraglutide API and Victoza® IC₅₀ dose on NO production in RAW 264.7 cell line model in LPS-stimulated conditions was calculated. We confirmed that the ten tested Liraglutide pre-formulations 1 to 10 have overall the same anti-inflammatory effect as Victoza®.

The in vitro efficacy of pre-formulations 1 to 10 is demonstrated, with an average IC₅₀ value of 48 nM to 61 nM.

### Example 3: In Vitro Efficacy of Pre-Formulations 2 and 6 of Example 2 on Murine Primary Chondrocytes

### Test System

Murine primary chondrocytes derived from C57Bl/6 newborn mice.

### Test Items and Test Items Vehicle

TABLE 7

| Pre- | | | Liraglutide |
|---|---|---|---|
| formulation | | | API |
| name | Isotonic agent | Buffer agent | (6 mg/ml) |
| Pre- | Glucose | Tromethamine | yes |
| formulation 2 | | | |
| Pre- | PEG400 | Tromethamine | yes |
| formulation 6 | | | |

Pre-formulations 2 and 6 are identical to pre-formulations respectively 2 and 6 of example 1.

### Formulations:

Interleukine-1β (IL-1β) (PeproTech) supplied as a powder was dissolved in water as a stock solution at 0.1 mg/ml. The concentrated stock solution was then diluted at final concentration (2 ng/ml) into the medium containing supplements (P/S, BSA, +/−Liraglutide).

Pre-formulations of Liraglutide (2 and 6) supplied as solutions at 6 mg/ml were diluted in PBS as a stock solution. The concentrated stock solution was then diluted in DMEM medium to reach the final concentrations of 3 μM, 1 μM, 333.3 nM, 111.1 nM, 37.0 nM, 12.3 nM and 4.1 nM.

Victoza® is a commercial Liraglutide drug. Test item supplied as a solution at 6 mg/ml was diluted in PBS as a stock solution. The concentrated stock solution was then diluted in DMEM medium to reach the final concentrations of 3 μM, 1 μM, 333.3 nM, 111.1 nM, 37.0 nM, 12.3 nM and 4.1 nM. Victoza® and the test items obtained from Victoza® are compositions according to the invention, as they comprise a GLP-1R agonist (liraglutide), a phosphate buffer and propylene glycol as isotonic agent.

The vehicle (PBS) is supplied “ready to use” and were diluted in cell seeding medium at a final concentration of 1:100.

### Experimental Design

### Isolation of Murine Articular Cartilage

Immature murine chondrocytes were derived from newborn pups (5-6 days old C57Bl/6). Joint was cleaned from the surrounding tissue with a scalpel, then it was cut in half to separate the two spheres, and then cut in half again. This allows for easier digestion. Femoral heads and condyles and tibial plateau were placed also in 30 ml of 1×PBS.

### Isolation of Immature Murine Chondrocytes

Pieces of cartilage were incubated twice in 10 ml of digestion solution (DMEM, 2 mM L-Glutamine, 1 g/L of glucose+1% P/S+Collagenase 3 mg/ml) for 45 min in an incubator at 37° C. with 5% CO₂ in a Petri dish 100 mm. Between the two digestions, pieces of cartilage were retrieved and placed in a new Petri dish. After the two digestions, dispersion of the aggregates was performed to obtained a suspension of isolated cells. Pieces of cartilage were incubated in 10 ml DMEM, 2 mM L-Glutamine, 1 g/L of glucose+1% P/S with collagenase D solution at 0.5 mg/ml (diluted to ⅙) overnight in an incubator at 37° C. with 5% CO₂.

### Seeding Chondrocytes

After overnight digestion, 10 ml of DMEM, 2 mM L-Glutamine, 1 g/L of glucose+10% FBS+1% P/S were added to a Petri dish to stop the collagenase D action. After dispersion of the aggregates with decreasing pipette sizes, a suspension of isolated cells was obtained and filtered through a sterile 70 μm cell strainer. Then, the cells were centrifuged for 10 min at 400 g at 20° C. The medium was removed and the pellet was resuspended in 5 ml of PBS to wash the cells. The cells were centrifuged for 10 min at 400 g at 20° C., and the PBS was replaced by 15 ml of DMEM 2 mM L-Glutamine, 1 g/L of glucose+10% FBS+1% P/S. The chondrocytes were counted in a Neubauer hemocytometer and observed to assess the viability of extracted cells. Chondrocytes were seeded at a density of 40×10³ cells in 2 ml of DMEM 2 mM L-Glutamine, 1 g/L of glucose+10% FBS+1% P/S per well in 12-well plates. The culture was maintained under sterile conditions in an incubator at 37° C. with 5% CO₂.

### Culture of Chondrocytes

Immature murine articular chondrocytes are expected to reach confluence by 6-7 days of culture. The culture medium was changed after 3 days of culture. At day 7, the DMEM medium containing 10% FBS was removed, the wells were rinsed twice with 1 ml of PBS and 1 ml of DMEM, 2 mM L-Glutamine, 1 g/L of glucose+1% P/S+0.1% BSA was added. At day 8, the medium was removed. 500 μl of fresh DMEM 2 mM L-Glutamine, 1 g/L of glucose+1% P/S+0.1% BSA containing 2 ng/ml of IL-10±Liraglutide pre-formulation or Victoza® were added in each well according to Table 8. The plates were incubated at 37° C.+5% CO₂ for 24 hours.

TABLE 8
Study design

| Group | Treatment | Induction | Time |
|---|---|---|---|
| 1 | Cells only (Blank) | Vehicle | 24 hours |
| | | (Water) | co-treatment |
| 2 | Vehicle (PBS) | IL-1β 2 ng/ml | |
| 3 | Pre-formulation 2 4.1 nM | | |
| 4 | Pre-formulation 2 12.3 nM | | |
| 5 | Pre-formulation 2 37.0 nM | | |
| 6 | Pre-formulation 2 111.1 nM | | |
| 7 | Pre-formulation 2 333.3 nM | | |
| 8 | Pre-formulation 2 1 μM | | |
| 9 | Pre-formulation 2 3 μM | | |
| 10 | Pre-formulation 6 4.1 nM | | |
| 11 | Pre-formulation 6 12.3 nM | | |
| 12 | Pre-formulation 6 37.0 nM | | |
| 13 | Pre-formulation 6 111.1 nM | | |
| 14 | Pre-formulation 6 333.3 nM | | |
| 15 | Pre-formulation 6 1 μM | | |
| 16 | Pre-formulation 6 3 μM | | |
| 17 | Victoza ® 4.1 nM (in PBS) | | |
| 18 | Victoza ® 12.3 nM (in PBS) | | |
| 19 | Victoza ® 37.0 nM (in PBS) | | |
| 20 | Victoza ® 111.1 nM (in PBS) | | |
| 21 | Victoza ® 333.3 nM (in PBS) | | |
| 22 | Victoza ® 1 μM (in PBS) | | |
| 23 | Victoza ® 3 μM (in PBS) | | |

Each condition treatment was run in quadruplicate.

TABLE 9
Study schedule

| | Study day | | | | | | |
|---|---|---|---|---|---|---|---|
| | Before | | | | | | After |
| Procedure | study | 1 | 4 | 7 | 8 | 9 | study |
| Isolation of murine | √ | | | | | | |
| articular cartilage | | | | | | | |
| Isolation of | √ | | | | | | |
| immature murine | | | | | | | |
| chondrocytes | | | | | | | |
| Plating of immature | | √ | | | | | |
| murine | | | | | | | |
| chondrocytes | | | | | | | |
| Change medium | | | √ | | | | |
| Change medium | | | | √ | | | |
| containing BSA | | | | | | | |
| Stimulation with | | | | | √ | | |
| IL-1β +/− Pre- | | | | | | | |
| formulation | | | | | | | |
| Victoza ® or | | | | | | | |
| vehicle | | | | | | | |
| Collect the | | | | | | √ | |
| supernatant | | | | | | | |
| Dosages (NO) | | | | | | | √ |

### Tests and Evaluations

At study termination, culture medium (±500 μl) of each well was collected in 1.5 ml tube (1 tube per well), centrifuged at 4000 rpm at room temperature and supernatant were added to a new 1.5 ml tube. Samples were kept at 2-8° C. for 2-3 days or frozen at −70° C. if Griess reagent Assay was not performed the same day.

As expected, NO production in vehicle conditions was below the limit of detection and were attributed a value of 1.56 μM. In IL-10-stimulated conditions, detection of nitrite oxide in culture medium is significantly increased compared to the vehicle. This secretion was significantly reduced by each tested-dose of Liraglutide pre-formulations or Victoza® compared to the IL-10 alone condition, with a sigmoidal dose response pattern. IC₅₀ was calculated for each formulation using Prism software and summary results are presented in Table 10.

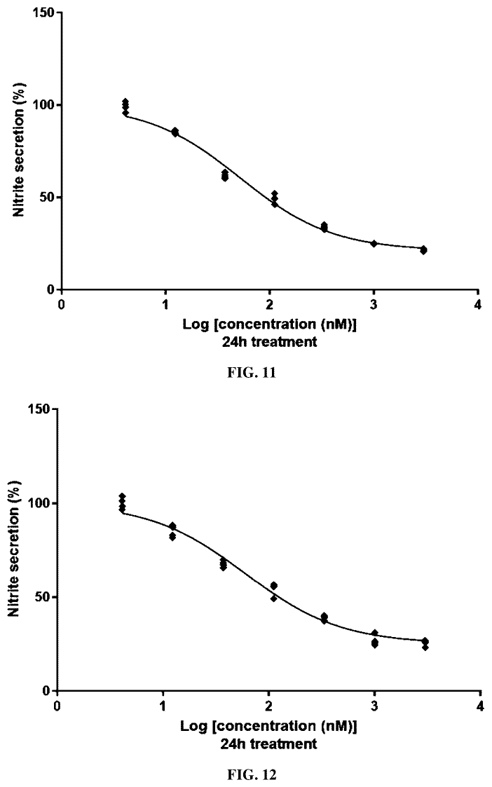

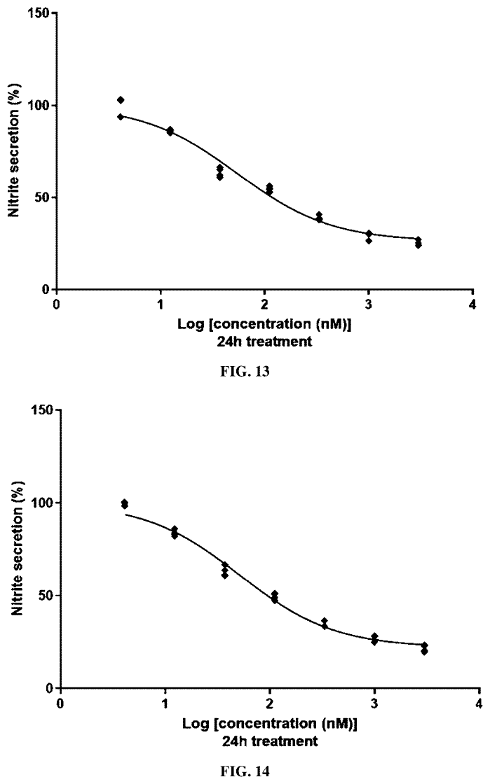

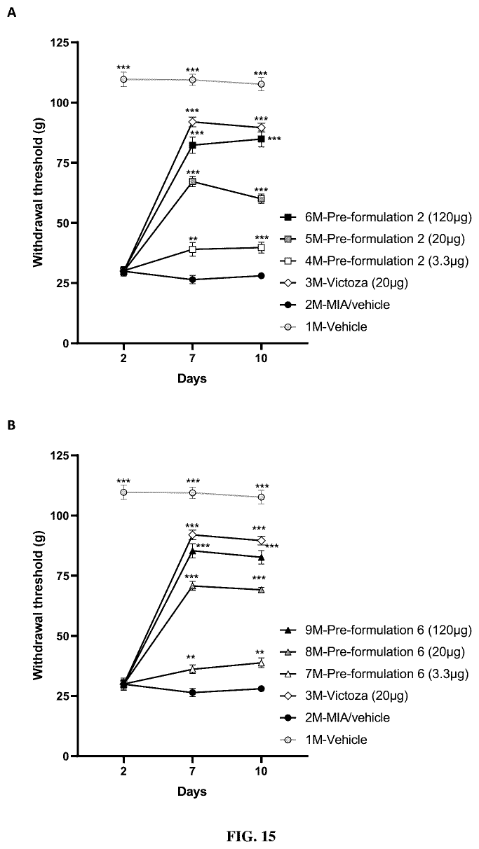

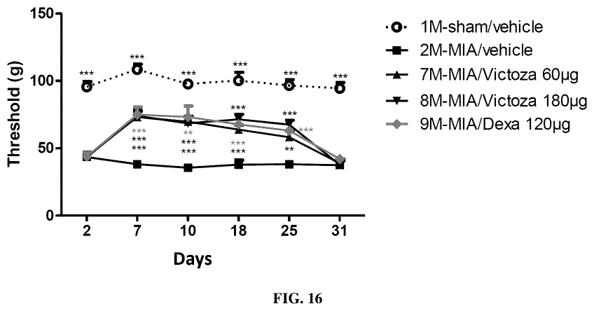

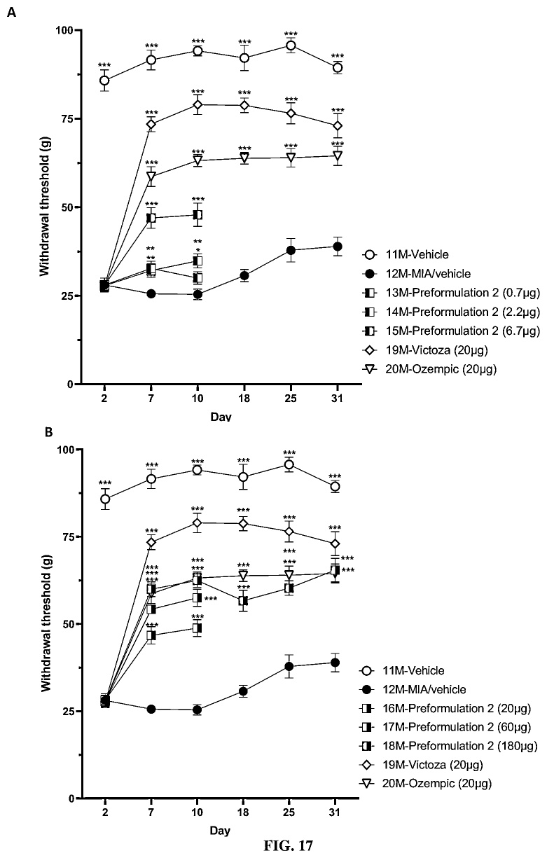

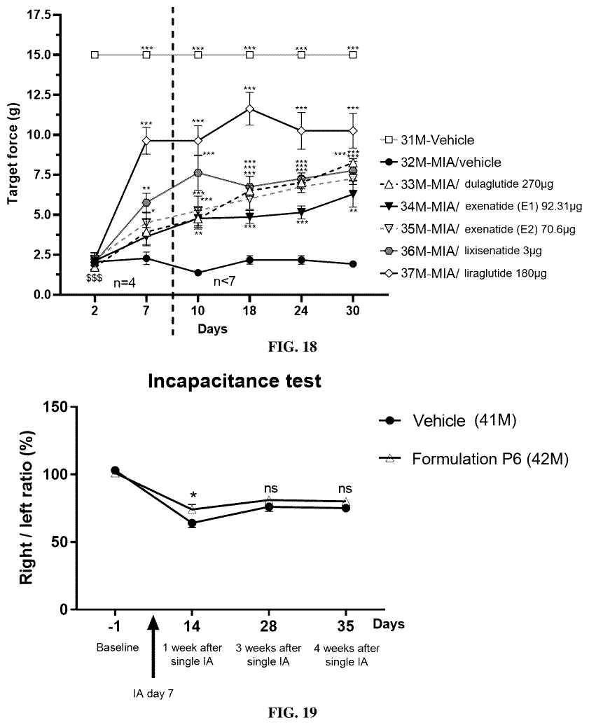

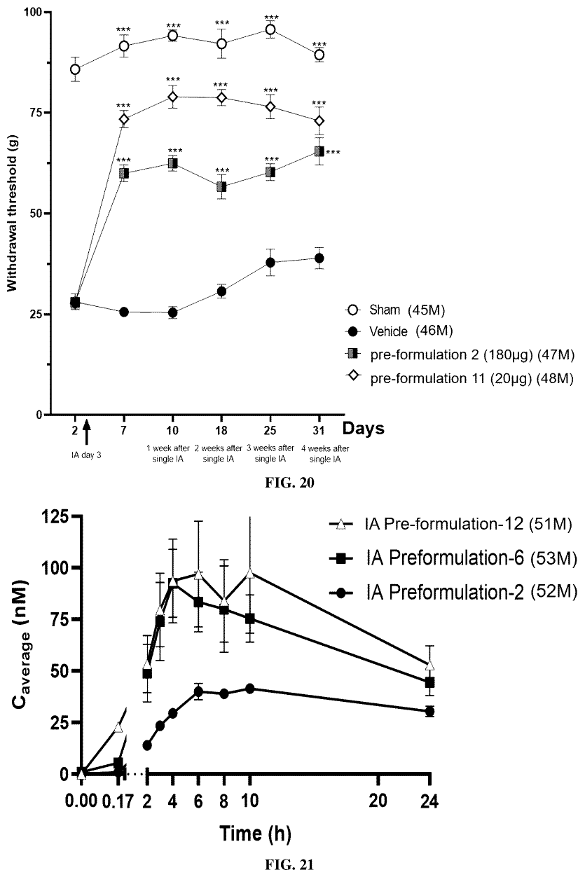

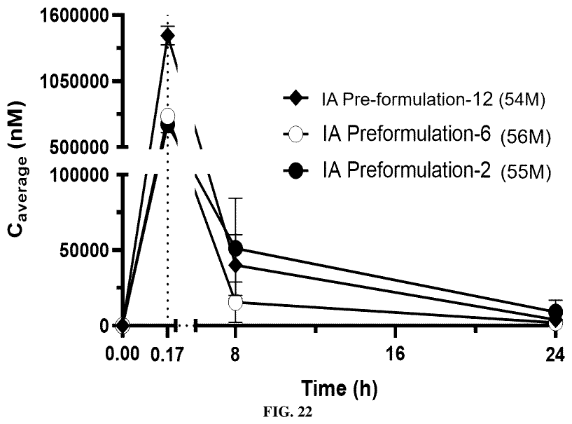

For murine primary chondrocytes cultured in low glucose conditions, Liraglutide pre-formulation 2 IC₅₀ dose on NO secretion is 60 nM with a confidence interval between 53 nM and 69 nM (FIG. 12); Liraglutide pre-formulation 6 IC₅₀ dose on NO secretion is 55 nM with a confidence interval between 46 nM and 64 nM (FIG. 13) and Victoza® IC₅₀ dose on NO secretion is 52 nM with a confidence interval between 46 nM and 59 nM (FIG. 14).

TABLE 10
IC₅₀ summary table

| Formulations according | |
|---|---|
| to the invention | IC50 value |
| Victoza ® (PBS) | 52 nM (46-59) |
| Pre-formulation 2 | 60 nM (53-69) |
| Pre-formulation 6 | 55 nM (46-64) |

### Conclusion

The objective of the study was to test 7 doses of 2 selected Liraglutide pre-formulations 2 and 6 in comparison to Victoza®, a marketed Liraglutide drug (4.1 nM to 3 μM) in murine primary chondrocytes.

We demonstrated that the compositions according to the invention (i.e. Liraglutide pre-formulations 2 and 6, and Victoza® test items), were able to dose-dependently inhibit the IL-10-induced NO production in murine primary chondrocytes. Liraglutide pre-formulations 2 and 6 and Victoza® IC₅₀ dose on NO production in murine primary chondrocytes model in IL-10-stimulated conditions was calculated. We confirmed that the two tested Liraglutide pre-formulations have overall the same anti-inflammatory and anti-degradative effect as Victoza®.

The in vitro efficacy of compositions 2 and 6 is demonstrated, with an average IC₅₀ value of 52 nM to 60 nM.

### Example 4: Efficacy of a Single Intra-Articular (IA) Knee Injection of 3 Ascending Doses of Preformulations-2 and -6 Utilizing a MIA-Induced Model of Osteoarthritis and Inflammatory Pain in Rats

### Test System

Sprague Dawley Rat.

### Test Items

TABLE 11

| Pre- formulation | Tonicity | | Liraglutide API |
|---|---|---|---|
| name | modifier | Buffer agent | (6 mg/ml) |
| Pre-formulation 2 | Glucose | Tromethamine | yes |
| Pre-formulation 6 | PEG400 | Tromethamine | yes |

Pre-formulations 2 and 6 are identical to pre-formulations respectively 2 and 6 of example 1.

### Formulations:

Pre-formulations of Liraglutide (2 and 6) supplied as solutions at 6 mg/ml were diluted in PBS to reach the final concentrations of 3,3 μg, 20 μg and 120 μg in 30 μL.

Victoza® is a commercial Liraglutide drug. Test item supplied as a solution at 6 mg/ml was diluted in PBS to reach the final concentrations of 20 μg in 30 μL. Victoza® and the test item obtained from Victoza® are compositions according to the invention, as they comprise a GLP-1R agonist (liraglutide), a phosphate buffer and propylene glycol as isotonic agent.

The vehicle (PBS) is supplied “ready to use” and were diluted in cell seeding medium at a final concentration of 1:100.

### Experimental Design

### OA Induction by Intra-Articular (IA) Injection of MIA (Monosodium Iodoacetate)

Animals were anesthetized via a chamber induction technique using inhalation anesthesia (Isoflurane at 5%). During the procedure, the animal was maintained under Isoflurane at a level between 1.5 and 3% with an air flow rate of 1-2 liters/minute. After induction of anesthesia, the right hind limb skin surface was clipped free of hair using electric animal clippers. After shaving, the area surrounding the knee joint was wiped with alcohol. A volume of 30 μL containing 3 mg MIA (monosodium iodoacetate) was injected intra-articularly (IA) in the knee joint through the patellar tendon. A 29-Gauge, 0.5-inch needle that was fitted with cannulation tubing was used such that only 3 to 4 mm of the needle was allowed to puncture the joint. After injection, the knee was massaged to ensure even distribution of the solution. Animals were injected once on day 1 (groups 2M, 3M, 4M, 5M, 6M, 7M, 8M, 9M). For group 1M (sham control), 30 μL of injectable saline was injected into knee joint.

### Mechanical Pain Evaluation (Von Frey Test)

von Frey test in the rats following MIA (monosodium iodoacetate) injection was conducted according to 4P-Pharma's Standard Operating Procedures (SOP). Rats were placed individually in the designated transparent Plexiglas chambers and allowed to acclimate for 10-15 min. Withdrawal responses to mechanical stimulation were determined using electronic von Frey apparatus (BIO-EVF5, Bioseb) with tip of 0.5 mm diameter on the stimulator handle applied from underneath the cage through openings (12×12 mm) in the plastic mesh floor. The withdrawal response in gram-force for each rat was calculated from three trials. Von Frey Test was performed on the animals at day 2 (as baseline before treatment, for group allocation), day 7 and day 10 (4 and 7 days following treatment, respectively): total of three times. The experimenter(s) was/were blind regarding the identity of the groups.

TABLE 12
Group allocation

| | | MIA | | | | | | |
|---|---|---|---|---|---|---|---|---|
| | | induction | | | | | | |
| | | (3 mg in | | Dose | | Dose | | Dose |
| Group | N= | 30 μL) | Treatment | level | | volume | ROA | regimen |
| 1M | 8 | — | Vehicle (PBS) | — | | 30 μL | IA | Once on |
| 2M | 8 | √ | Vehicle (PBS) | — | | | | day 3 |
| 3M | 8 | √ | Victoza ® | 20 | μg | | | |
| 4M | 8 | √ | Pre-formulation 2 | 3.3 | μg | | | |
| 5M | 8 | √ | | 20 | μg | | | |
| 6M | 8 | √ | | 120 | μg | | | |
| 7M | 8 | √ | Pre-formulation 6 | 3.3 | μg | | | |
| 8M | 8 | √ | | 20 | μg | | | |
| 9M | 8 | √ | | 120 | μg | | | |

TABLE 13
Study timeline

| Study | | |
|---|---|---|
| Day/week* | Procedure | Remarks |
| D 1 | MIA injection, 3 mg in 30 μL, IA | Except for group 1M (sham control), |
| | | 30 μL injectable saline, IA |
| D 3 | Treatment (Vehicle, Victoza ® or | IA single injection, 30 μL |
| | Liraglutide pre-formulations | |
| | 2 or 6) | |
| D 2, D 7, D 10 | von Frey test | |
| D 11 | Termination | Right knee (diseased) collection |

### Statistical Plan

For statistics, we applied a sequential testing strategy reflecting experimental expectations, while retaining the central Mann-Whitney (non-parametric) test. Sequential statistics were made to compare in this order:
1. The control/vehicle group (1M) and the MIA/vehicle groups (2M).
2. The MIA/vehicle groups (2M) and the MIA/Victoza®-treated animals (20 μg) (3M).
3. The MIA/vehicle groups (2M) and the higher dose of preformulation-2 or -6 (120 μg) (6M and 9M). Subsequently, we compare the MIA/vehicle group (2M).

### Tests and Evaluations

### Mechanical Pain Evaluation (Von Frey Test)

Allodynia in rats following OA induction on day 1 was evaluated on day 2 as baseline for group allocation and then on days 7 and 10 to assess effect of treatments on mechanically-induced pain. For each group, mean of withdrawal responses, as given by the electronic von Frey apparatus, for the left and right hind paws, was calculated. Group average animal withdrawal response results are presented in Table 14 for the right hind paw. Results on the right hind paw are presented in FIG. 15. FIG. 15A compares vehicle control, MIA/vehicle and MIA/Victoza®-groups (1M-3M) to the three groups of Preformulation-2 (4M-6M), FIG. 15B compares vehicle control, MIA/vehicle and MIA/Victoza®-groups (1M-3M) to the three groups of Preformulation-6 (7M-9M).

On day 7, there was a significant increase of paw withdrawal threshold in MIA-injected animals between Victoza®-treated group 3M vs MIA/vehicle group 2M (mean±SD; p<0.001). A significant dose effect was observed in preformulations groups at 120 μg (9M and 6M), 20 μg (8M and 5M), and 3,3 μg (7M and 4M) vs MIA/vehicle group 2M (mean±SD; p<0.001 for 120 and 20 μg doses and mean±SD; p<0.01 for 3,3 μg dose) on day 7.

A significant decrease on the withdrawal threshold from D7 to D10 was observed using the 20 μg of preformulation-2 (5M) (mean±SD; p<0.05). The withdrawal threshold effect lasted from D7 to D10 using the preformulation-2 at 3,3 and 120 μg (4M and 6M) and the preformulation-6 (7M, 8M and 9M) since non-statistical difference was observed from D7 to D10 on these groups (4M, 6M, 7M, 8M and 9M). Higher paw withdrawal threshold was observed when comparing 20 μg of Victoza® IA injection and of 20 μg of preformulation-2 and -6.

TABLE 14
Summarized averages of right paw withdrawal threshold in grams (mean ± SD)

| | | D2 | | D7 | | D10 | |
|---|---|---|---|---|---|---|---|
| Group | Treatment | Mean | SD | Mean | SD | Mean | SD |
| 1M | Vehicle | 109.62*** | 8.37 | 109.43*** | 6.54 | 107.62*** | 7.96 |
| 2M | MIA/Vehicle | 29.85 | 5.63 | 26.45 | 4.74 | 28.00 | 3.13 |
| 3M | MIA/Victoza | 29.92 | 3.65 | 91.93*** | 5.62 | 89.56*** | 5.13 |
| | (20 μg) | | | | | | |
| 4M | MIA/Preformulation- | 30.09 | 5.09 | 38.98** | 8.01 | 39.71*** | 6.38 |
| | 2 (3.3 μg) | | | | | | |
| 5M | MIA/Preformulation- | 29.93 | 4.51 | 67.20*** | 6.07 | 60.05*** | 5.43 |
| | 2 (20 μg) | | | | | | |
| 6M | MIA/Preformulation- | 30.07 | 5.15 | 82.24*** | 9.52 | 84.88*** | 9.42 |
| | 2 (120 μg) | | | | | | |
| 7M | MIA/Preformulation- | 29.99 | 4.32 | 36.10** | 4.99 | 38.78** | 5.60 |
| | 6 (3.3 μg) | | | | | | |
| 8M | MIA/Preformulation- | 30.10 | 7.19 | 70.72*** | 5.28 | 69.13*** | 2.93 |
| | 6 (20 μg) | | | | | | |
| 9M | MIA/Preformulation- | 29.75 | 5.32 | 85.31*** | 8.36 | 82.64*** | 7.99 |
| | 6 (120 μg) | | | | | | |

MIA/Vehicle (2M) vs Control/Vehicle (1M) (Mann-Whitney test; ***p < 0.001).
MIA/Vehicle (2M) vs MIA preformulation-2 and -6 (4M, 5M, 6M, 7M, 8M and 9M) (Mann-Whitney test; **p < 0.01, ***p < 0.001).

### Conclusion

In conclusion, in this comparative study between IA injection of 20 μg of Victoza® and three doses (3.3, 20 and 120 μg) of preformulation-2 and -6 we observed that both preformulations 2 and 6 display dose-dependent analgesic effects.

### Example 5: Efficacy of Intra-Articular (IA) Knee Injection of Victoza® Utilizing a MIA-Induced Model of Osteoarthritis and Inflammatory Pain in Rats

### Objective:

The objective of the present study was double: to perform a dose response study using Victoza® utilizing a MonoIodoAcetate (MIA)-induced model of osteoarthritis and inflammatory pain in rats to calculate a half maximal effective concentration EC50 (short term MIA model) and to determine on 5 selected groups the duration of the analgesic effect after a single intra-articular administration of Victoza® (long term MIA model). Dexamethasone was used as a reference positive-control group in the study.

### Method:

Nine groups comprising 8 SD rats per group were allocated for this study performed in 4 cycles. Chemically-induced OA disease was performed on eight out of nine groups. On day 1, 3 mg of MIA in 30 μL was injected intra-articularly (IA) in the right knee joint. The last group (sham control) received 30 μL of saline IA in the right knee joint. The nine groups were as follows: one sham control group (1M) treated with vehicle (water) and eight MIA groups treated with vehicle (2M), Victoza® at 0.7 μg (3M), 2.2 μg (4M), 6.7 μg (5M), 20 μg (6M), 60 μg (7M) or 180 μg (8M) and dexamethasone at 120 μg (9M). Victoza® and the test item obtained from Victoza® are compositions according to the invention, as they comprise a GLP-1R agonist (liraglutide), a phosphate buffer and propylene glycol as isotonic agent. All animals were injected IA once on day 3. During the study, mortality and morbidity observation and von Frey tests using electronic von Frey apparatus were performed. Group allocation was performed on day 2, based on von Frey results. Knee harvesting was performed for optional analyses at study termination (day 11 for short term MIA model or day 32 for long term MIA model).

### Results:

### EC50, Short Term MIA Model (Up to Day 11) for all Groups:

Mortality/morbidity: No mortality nor morbidity was observed during the study.

Von Frey test: As expected, there was a significant decrease of paw withdrawal threshold following MIA injection as compared to sham control (group 1M), confirming model induction. This effect lasted until study termination for group 2M. On day 7, a significant dose-dependent increase in paw withdrawal threshold was observed between MIA/Victoza®-treated groups 5M, 6M, 7M and 8M as well as group 9M treated with dexamethasone 120 μg vs MIA/vehicle group 2M. Calculated EC50 was 3.3 μg. On day 10, a significant dose-dependent increase in paw withdrawal threshold was observed between MIA/Victoza®-treated groups 4M, 5M, 6M, 7M and 8M as well as group 9M (dexamethasone) vs MIA/vehicle group 2M. Calculated EC50 was 2.1p g.

### Long Term MIA Model (Up to Day 32) for 5 Selected Groups:

The 5 selected groups were: 1M (sham/vehicle), 2M (MIA/vehicle), 7M (MIA/Victoza® 60 μg), 8M (MIA/Victoza® 180 μg) and 9M (MIA/dexamethasone).

Mortality/morbidity: No mortality or morbidity was observed during the study.

Von Frey test: After day 10 (i.e. one week after acute treatment), von Frey tests were performed once a week at day 18, 25, 31 until study termination. The significant decrease of paw withdrawal threshold following MIA injection lasted until study termination for group 2M as compared to sham control group 1M, confirming model induction and maintenance up to day 31. For the other groups, results indicated that the analgesic effect of Victoza® 60 μg (group 7M) and 180 μg (group 8M) as well as the one of dexamethasone 120 μg (group 9M) was maintained significantly up to day 25 (i.e. 3 weeks following acute administration) but was lost at day 31 (i.e. 4 weeks following acute injection). Results are presented in FIG. 16.

### Conclusion

In conclusion, this study confirms that locally-administered a composition according to the invention (for example Victoza®) targets relevant mechanisms associated with pain in a MIA-induced OA and inflammatory pain model in rats. The calculated half maximal effective concentration is 2.1 μg at day 7 and 3.3 μg at day 10. Moreover, here, we demonstrate a 4 weeks long-lasting analgesic effect of a composition according to the invention (for example Victoza®) following acute IA administration comparable to that of dexamethasone positive control.

### Example 6: Efficacy of a Single Intra-Articular (IA) Knee Injection of Ozempic® and of 6 Ascending Doses of Pre-Formulation 2 Utilizing a MIA-Induced Model of Osteoarthritis and Inflammatory Pain in Rats

### Test System

Sprague Dawley Rat.

Test items

TABLE 15

| Pre- formulation | Tonicity | | Liraglutide API |
|---|---|---|---|
| name | modifier | Buffer agent | (6 mg/ml) |
| Pre-formulation 2 | Glucose | Tromethamine | yes |

Pre-formulation 2 is identical to pre-formulation 2 of example 1.

### Formulations:

Pre-formulation 2 of Liraglutide supplied as solution at 6 mg/ml was diluted in PBS to reach the final concentrations of 0.7 μg, 2.2 μg, 6.7 μg, 20 μg, 60 μg and 180 μg in 30 μL.

Victoza® is a commercial Liraglutide drug. Test item supplied as a solution at 6 mg/ml was diluted in PBS to reach the final concentration of 20 μg in 30 μL. Victoza® and the test item obtained from Victoza® are compositions according to the invention, as they comprise a GLP-1R agonist (liraglutide), a phosphate buffer and propylene glycol as isotonic agent.

Ozempic® is a commercial Semaglutide drug. Test item supplied as a solution at 1.34 mg/ml was diluted in PBS to reach the final concentration of 20 μg in 30 μL.

The vehicle (PBS) is supplied “ready to use” and were diluted in cell seeding medium at a final concentration of 1:100.

### Experimental Design

### OA Induction by Intra-Articular (IA) Injection of MIA (Monosodium Iodoacetate)

Animals were anesthetized via a chamber induction technique using inhalation anesthesia (Isoflurane at 5%). During the procedure, the animal was maintained under Isoflurane at a level between 1.5 and 3% with an air flow rate of 1-2 liters/minute. After induction of anesthesia, the right hind limb skin surface was clipped free of hair using electric animal clippers. After shaving, the area surrounding the knee joint was wiped with alcohol. A volume of 30 μL containing 3 mg MIA (monosodium iodoacetate) was injected intra-articularly (IA) in the knee joint through the patellar tendon. A 29-Gauge, 0.5-inch needle that was fitted with cannulation tubing was used such that only 3 to 4 mm of the needle was allowed to puncture the joint. After injection, the knee was massaged to ensure even distribution of the solution. Animals were injected once on day 1 (groups 12M, 13M, 14M, 15M, 16M, 17M, 18M, 19M and 20M). For group 11M (sham control), 30 μL of injectable saline was injected into knee joint.

### Mechanical Pain Evaluation (Von Frey Test)

Von Frey test in the rats following MIA (monosodium iodoacetate) injection was conducted according to 4P-Pharma's Standard Operating Procedures (SOP). Rats were placed individually in the designated transparent Plexiglas chambers and allowed to acclimate for 10-15 min. Withdrawal responses to mechanical stimulation were determined using electronic von Frey apparatus (BTO-EVF5, Bioseb) with tip of 0.5 mm diameter on the stimulator handle applied from underneath the cage through openings (12×12 mm) in the plastic mesh floor. The withdrawal response in gram-force for each rat was calculated from three trials, von Frey Test was performed on the animals at day 2 (as baseline before treatment, for group allocation), day 7, day 10, day 18, day 25 and day 31 (4, 7, 15, 22 and 28 days following treatment, respectively): total of six times. The experimenter(s) was/were blind regarding the identity of the groups.

TABLE 16
Group allocation

| | | MIA | | | | | | |
|---|---|---|---|---|---|---|---|---|
| | | induction | | | | | | |
| | | (3 mg in | | Dose | | Dose | | Dose |
| Group | N= | 30 μL) | Treatment | level | | volume | ROA | regimen |
| 11M | 8 | — | Vehicle (PBS) | — | | 30 μL | IA | Once on |
| 12M | 8 | √ | Vehicle (PBS) | — | | | | day 3 |
| 13M | 8 | √ | Pre-formulation 2 | 0.7 | μg | | | |
| 14M | 8 | √ | | 2.2 | μg | | | |
| 15M | 8 | √ | | 6.7 | μg | | | |
| 16M | 8 | √ | | 20 | μg | | | |
| 17M | 8 | √ | | 60 | μg | | | |
| 18M | 8 | √ | | 180 | μg | | | |
| 19M | 8 | √ | Victoza ® | 20 | μg | | | |
| 20M | 8 | √ | Ozempic ® | 20 | μg | | | |

N = number of animals.

TABLE 17
Study timeline

| Study | | |
|---|---|---|
| Day/week* | Procedure | Remarks |
| D 1 | MIA injection, 3 mg in 30 μL, IA | Except for group 11M (sham |
| | in the right knee | control): 30 μL injectable saline, IA |
| D 3 | IA Treatment (Vehicle, | IA single injection, 30 μL, right knee |
| | Victoza ®, Ozempic ® or | |
| | Liraglutide pre-formulation) | |
| D 2, D 7, D 10 | Von Frey test for all groups | |
| D 11 | Termination for groups 13M, | Right knee (diseased) collection |
| | 14M, 15M, 16M and 17M | |
| D 18, D 25, D 31 | von Frey test for groups 11M, | |
| | 12M, 18M, 19M and 20M | |
| D 31 | Termination for groups 11M, | Right knee (diseased) collection |
| | 12M, 18M, 19M and 20M | |

### Statistical Plan

For statistics, we applied a sequential testing strategy reflecting experimental expectations, while retaining the central Mann-Whitney (non-parametric) test. Sequential statistics were made to compare in this order:
1. The control/vehicle group (11M) and the MIA/vehicle groups (12M).
2. The MIA/vehicle groups (12M) and the MIA/Victoza®-treated animals (20 μg) (19M).
3. The MIA/vehicle groups (12M) and the higher dose of pre-formulation 2 (180 μg) (18M). Subsequently, we compare the MIA/vehicle group (12M) and the MIA/Ozempic®-treated animals (20 μg) (20M).

### Tests and Evaluations

### Mechanical Pain Evaluation (Von Frey Test)

Allodynia in rats following OA induction on day 1 was evaluated on day 2 as baseline for group allocation and then on days 7, 10, 18, 25 and 31 to assess effect of treatments on mechanically-induced pain. For each group, mean of withdrawal responses, as given by the electronic von Frey apparatus, for the left and right hind paws, was calculated. Group average animal withdrawal response results are presented in Table 18A and Table 18B for the right hind paw. Results on the right hind paw are presented in FIG. 17. FIG. 17A compares vehicle control, MIA/vehicle, MIA/Victoza® and MIA/Ozempic®-groups (11M, 12M, 19M, 20M) to three of the six groups of Preformulation-2 (13M-15M), FIG. 17B compares vehicle control, MIA/vehicle, MIA/Victoza® and MIA/Ozempic®-groups (11M, 12M, 19M, 20M) to the three other groups of Preformulation-2 (16M-18M).

On day 7, day 10, day 18, day 25 and on day 31 there was a significant increase of paw withdrawal threshold in MIA-injected animals between Victoza®-treated group 19M vs MIA/vehicle group 12M (mean±SD; p<0.001). A significant dose effect was observed in preformulations groups (13M-18M) vs MIA/vehicle group 12M (mean±SD; p<0.001, p<0.01 and p<0.05) on day 7, day 10, day 18, day 25 and on day 31. A significant dose effect was observed in Ozempic®-treated group (20M) vs MIA/vehicle group 12M (mean±SD; p<0.001) on day 7, day 10, day 18, day 25 and on day 31. There was no significant difference in paw withdrawal threshold between all treated groups (groups 13M to 20M).

TABLE 18A
Summarized averages of right paw withdrawal threshold
in grams at D2, D7 and D10 (mean ± SD)

| | | D2 | | D7 | | D10 | |
|---|---|---|---|---|---|---|---|
| Group | Treatment | Mean | SD | Mean | SD | Mean | SD |
| 11M | Control/Vehicle | 85.80*** | 8.43 | 91.59*** | 7.88 | 94.16*** | 3.96 |
| 12M | MIA/Vehicle | 28.08 | 5.49 | 25.55 | 3.26 | 25.40 | 4.15 |
| 13M | Preformulation | 28.03 | 5.53 | 32.69** | 5.84 | 30.03* | 4.94 |
| | 2 (0.7 μg) | | | | | | |
| 14M | Preformulation | 27.79 | 3.56 | 32.11** | 5.38 | 34.84** | 5.58 |
| | 2 (2.2 μg) | | | | | | |
| 15M | Preformulation | 27.99 | 3.86 | 46.95*** | 8.29 | 47.85*** | 9.32 |
| | 2 (6.7 μg) | | | | | | |
| 16M | Preformulation | 28.19 | 3.46 | 46.73*** | 7.06 | 48.81*** | 6.89 |
| | 2 (20 μg) | | | | | | |
| 17M | Preformulation | 27.80 | 5.06 | 54.19*** | 3.15 | 57.50*** | 6.88 |
| | 2 (60 μg) | | | | | | |
| 18M | Preformulation | 27.96 | 2.83 | 59.99*** | 5.98 | 62.46*** | 5.51 |
| | 2 (180 μg) | | | | | | |
| 19M | Victoza ® | 27.70 | 3.48 | 73.43*** | 6.04 | 78.95*** | 7.87 |
| | (20 μg) | | | | | | |
| 20M | Ozempic ® | 27.71 | 4.05 | 58.64*** | 7.89 | 63.19*** | 4.85 |
| | (20 μg) | | | | | | |

TABLE 18B
Summarized averages of right paw withdrawal threshold
in grams at D18, D25 and D31 (mean ± SD)

| | | D18 | | D25 | | D31 | |
|---|---|---|---|---|---|---|---|
| Group | Treatment | Mean | SD | Mean | SD | Mean | SD |
| 11M | Control/Vehicle | 92.14*** | 10.16 | 95.71*** | 5.99 | 89.39*** | 4.96 |
| 12M | MIA/Vehicle | 30.68 | 4.89 | 37.85 | 9.39 | 38.91 | 7.50 |
| 13M | Preformulation | NA | NA | NA | NA | NA | NA |
| | 2 (0.7 μg) | | | | | | |
| 14M | Preformulation | NA | NA | NA | NA | NA | NA |
| | 2 (2.2 μg) | | | | | | |
| 15M | Preformulation | NA | NA | NA | NA | NA | NA |
| | 2 (6.7 μg) | | | | | | |
| 16M | Preformulation | NA | NA | NA | NA | NA | NA |
| | 2 (20 μg) | | | | | | |
| 17M | Preformulation | NA | NA | NA | NA | NA | NA |
| | 2 (60 μg) | | | | | | |
| 18M | Preformulation | 56.61*** | 8.53 | 60.28*** | 5.87 | 65.43*** | 9.50 |
| | 2 (180 μg) | | | | | | |
| 19M | Victoza ® | 78.75*** | 5.83 | 76.51*** | 8.41 | 72.99*** | 9.65 |
| | (20 μg) | | | | | | |
| 20M | Ozempic ® | 63.83*** | 4.65 | 63.95*** | 7.54 | 64.51*** | 7.69 |
| | (20 μg) | | | | | | |

MIA/Vehicle (12M) vs Control/Vehicle (11M) (Mann-Whitney test; ***p < 0.001).
MIA/Vehicle (12M) vs MIA preformulation-2, Victoza and Ozempic (14M, 15M, 16M, 17M, 18M, 19M and 20M) (Mann-Whitney test; *p < 0.05, **p < 0.01, ***p < 0.001).

### Conclusion

In conclusion, in this comparative study between IA injection of 20 μg of Victoza®, 20 μg of Ozempic® and six doses (0.7 μg, 2.2 μg, 6.7 μg, 20 μg, 60 μg and 180 μg) of preformulation-2, we observed that all preformulations-2 (i.e. compositions according to the invention) display dose-dependent analgesic effects. In addition, Victoza® (i.e. a composition according to the invention) and Ozempic® also display analgesic effects.

### Example 7: Evaluation of the Analgesic Effect of Various GLP-1R-Agonists Utilizing a MIA-Induced Model of Osteoarthritis and Inflammatory Pain in Rats

### Objective:

The objective of the present study was to evaluate the analgesic effect of the maximum feasible dose of the GLP-1R agonists Dulaglutide®, exenatide and lixisenatide, in liquid homogeneous compositions, in particular in solutions, compared to liraglutide in a liquid homogeneous composition, in particular in a solution, in MIA-induced model of osteoarthritis and inflammatory pain in rats.

### Method:

Seven groups comprising 8 SD rats per group were allocated for this study performed in 2 cycles (4 SD rats/cycle). Chemically-induced OA disease was performed on six out of seven groups by injecting intra-articularly (IA) in the right knee joint 3 mg of MIA in 30 μL on day 1. The control group (sham/vehicle control) received 30 μL of saline IA in the right knee joint. The seven groups were as follow: a sham/vehicle control group (group 31M) treated with vehicle (NaCl 0.9%) and six MIA groups treated with vehicle (group 32M), with 270 μg of dulaglutide (group 33M), 92.31 μg of exenatide (E1) (group 34M), 70.6 μg of exenatide (E2) (group 35M), 3 μg of Adlyxin (group 36M), and 180 μg of Victoza® (group 37M). All groups 31M-37M received 1 IA injection of either saline (group 31M) or MIA (groups 32M to 37M) on day 1 and the treatment injection on day 3. During the study, body weight measurements (twice a week) and von Frey tests using electronic von Frey apparatus (on day 2, 7, for C1) and von Frey filaments (on day 10, 18, 24 and 30 for Cl, and on day 2, 7, 10, 18, 24 and 30 for C2) were performed. Group allocation was performed on day 2, based on von Frey results. Knee harvesting was performed for optional analyses at study termination at day 32.

### Formulations:

NaCl 0.9% (vehicle) is supplied as “ready to use” for intra-articular injection (30 μL) for groups 31M and 32M.

A solution comprising dulaglutide is prepared from the product Trulicity®. Trulicity® is a commercial dulaglutide drug. Trulicity® is supplied as a “ready to use” solution at 4.5 mg/0.50 mL. It will be diluted in the appropriate volume of vehicle (NaCl 0.9%) for injection into knee joint of 270 μg in 30 μL per rat for group 33M.

A solution comprising exenatide (E1) is prepared from the product Bydureon®. Bydureon® is a commercial exenatide drug. Bydureon® is supplied as a “ready to use” solution at 2 mg/0.65 mL. It will be diluted in the appropriate volume of vehicle (NaCl 0.9%) for injection into knee joint of 92.31 μg in 30 μL per rat for group 34M.

Another solution comprising exenatide (E2) is prepared from the product Bydureon Bcise®. Bydureon Bcise® is a commercial exenatide drug with extended release. Bydureon Bcise® is supplied as a “ready to use” solution at 2.35 mg/mL. It will be diluted in the appropriate volume of vehicle (NaCl 0.9%) for injection into knee joint of 70.6 μg in 30 μL per rat for group 35M.

A solution comprising lixisenatide is prepared from the product Adlyxin®. Adlyxin® is a commercial lixisenatide drug. Adlyxin® is supplied as a “ready to use” solution at 100 μg/mL. It will be diluted in the appropriate volume of vehicle (NaCl 0.9%) for injection into knee joint of 3 μg in 30 μL per rat for group 36M.

A solution comprising liraglutide is prepared from the product Victoza®. Victoza® is a commercial liraglutide drug. Victoza® is supplied as a “ready to use” solution at 6 mg/mL. It will be diluted in the appropriate volume of vehicle (NaCl 0.9%) for injection into knee joint of 180 μg in 30 μL per rat for group 37M.

### Experimental Design

### OA Induction by Intra-Articular (IA) Injection of MIA (Monosodium Iodoacetate)

56 male SD rats aged 9 weeks were anesthetized via a chamber induction technique using inhalation anesthesia (Isoflurane at 5%). During the procedure, the animal was maintained under Isoflurane at a level between 1.5 and 3% with an air flow rate of 1-2 liters/minute. After induction of anesthesia, the right hind limb skin surface was clipped free of hair using electric animal clippers. After shaving, the area surrounding the knee joint was wiped with alcohol. A volume of 30 μL containing 3 mg MIA was injected intra-articularly (IA) in the knee joint through the patellar tendon. A 29-gauge, 0.5-inch needle that fitted with cannulation tubing was used such that only 3 to 4 mm of the needle was allowed to puncture the joint. After injection, the knee was massaged to ensure even distribution of the solution. Animals were injected once on day 1 (groups 32M, 33M, 34M, 35M, 36M, 37M). For group 31M (sham control), 30 μL of injectable saline was injected into knee joint.

The study was conducted in two cycles according to Table 19 for study design and Table 20 for study timeline. Group allocation was performed on day 2 from Von Frey.

TABLE 19
Group allocation

| | | MIA | | | | | | |
|---|---|---|---|---|---|---|---|---|
| | | induction | | | | | | |
| | | (3 mg in | | Dose | | Dose | Route of | Dose |
| Group | N= | 30 μL) | Treatment | level | | volume | administration | regimen |
| 31M | 8 | Injectable | Vehicle | — | | 30 μL | IA | Once on |
| | | saline | (NaCL 0.9%) | | | | | day 3 |
| 32M | 8 | √ | Vehicle | — | | | | |
| | | | (NaCL 0.9%) | | | | | |
| 33M | 8 | √ | Dulaglutide | 270 | μg | | | |
| 34M | 8 | √ | Exenatide (E1) | 92.31 | μg | | | |
| 35M | 8 | √ | Exenatide (E2) | 70.6 | μg | | | |
| 36M | 8 | √ | Lixisenatide | 3 | μg | | | |
| 37M | 8 | √ | Liraglutide | 180 | μg | | | |

N = number of animals.

TABLE 20
Study timeline

| Study Day/week | Procedure | Remarks |
|---|---|---|
| D 1 | MIA injection, 3 mg in 30 μL, IA, | Except for group 31M (sham |
| | in the right knee | control): 30 μL injectable saline, IA |
| D 3 | IA treatment (Vehicle, | IA single injection, 30 μL, right knee |
| | liraglutide, dulaglutide, | |
| | exenatide (E1), exenatide (E2), | |
| | and lixisenatide) | |
| D 2, D 7, D 10, D 18 | Von Frey test for all groups | |
| D 24 and D 30 | | |
| D 32 | Termination for all groups | Right knee (diseased) collection |

### Mechanical Pain Evaluation (Von Frey Test)

Von Frey test in the rats following MIA injection was conducted according to 4P-Pharma's proceedings “Mechanical Neuropathic Pain Evaluation (electronic Von Frey)” and to 4P-Pharma's proceedings “Mechanical Neuropathic Pain Evaluation (Von Frey)”. Rats were placed individually in the designated transparent Plexiglas chambers and allowed to acclimate for 10-15 min. Withdrawal response to mechanical stimulation was determined using electronic von Frey apparatus (BIO-EVF5, Bioseb) with a 0.5 mm diameter tip placed on the stimulator handle applied from underneath the cage through openings (12×12 mm) in the plastic mesh floor or with von Frey filaments (Bio-VF-M, Bioseb). The withdrawal response in gram-force for each rat was calculated from three trials for von Frey apparatus and once with von Frey filaments. Von Frey Test was performed on the animals at day 2 (as baseline before treatment, for group allocation), day 7 and day 10 (4- and 7-days following treatment, respectively). The experimenter(s) were blind regarding the identity of the groups.

### Statistical Plan

For statistics, we applied a sequential testing strategy reflecting experimental expectations, while retaining the central Mann-Whitney (non-parametric) test to evaluate the mechanical pain evaluation. First, we compared the difference between sham (group 31M) and MIA/vehicle (group 32M). As the difference was found significant, we then compared the difference between MIA/liraglutide treated animals (group 37M) with the MIA/vehicle (group 32M) animals. As the results were found significant, we tested the statistical difference between MIA/vehicle and the rest of GLP-1 analogue-treated animals (group 33M, 34M, 35M and 36M).

RESULTS: mechanical pain evaluation (von Frey test): evaluation of allodynia in long term MIA model

Allodynia in rats following OA induction on day 1 was evaluated on day 2 as baseline for group allocation and then on days 7, 10, 18, 24 and 30 to assess effect of treatments on mechanically induced pain. For each group, mean of withdrawal responses, as given by the electronic von Frey apparatus (for animals from Cycle 1 on day 2 and day 7) (Table 21) or the von Frey filaments (from day 7 for Cycle 1 and from day 2 for Cycle 2) (Tables 22 and 22bis, FIG. 18), was calculated for the right hind paws. FIG. 18 compares vehicle control and MIA/vehicle groups (31M, 32M) to treated-groups (33M-37M).

Individual animal withdrawal responses are presented in Table 21 for the right hind paw. MIA IA injection on day 1 resulted in a significant reduction on paw withdrawal threshold that was characterized on day 2 between saline group (31M) and the rest of the MIA groups (32M to 37M) (mean±SD; $p<0.005, ^{$$$}p<0.001). For group 32M, this effect lasted until study termination thus, validating the MIA IA injection and MIA rat model on the rapid pain-like responses in the ipsilateral limb in rats (FIG. 18).

Comparing only 4 animals per group and using the von Frey filaments technique on day 7, results indicated that there was a significant increase of paw withdrawal threshold in MIA-injected animals treated with 70.6 μg of exenatide (E2), 3 μg of lixisenatide and 180 μg of liraglutide (35M, 36M and 37M) compared to MIA/vehicle treated group (32M) (mean±SD; *p<0.05, **p<0.01, ***p<0.001) (FIG. 18). Upon day 10 until day 30, all treated groups (33M, 34M, 35M, 36M and 37M) displayed a significant increase of paw withdrawal threshold compared to MIA/vehicle treated group (32M) for each day (mean±SD; **p<0.01, ***p<0.001) (FIG. 18).

TABLE 21
Summarized averages of right paw withdrawal threshold
in grams measured by Electronic von Frey at
D2, D7 (mean ± SD, N) (cycle 1)

| | | D2 | | | D7 | | |
|---|---|---|---|---|---|---|---|
| Group | Treatment | Mean | SD | N | Mean | SD | N |
| 31M | Control/Vehicle | 98.83 | 11.33 | 4 | 98.31 | 8.45 | 4 |
| 32M | MIA/Vehicle | 32.60 | 7.72 | 4 | 29.55 | 4.01 | 4 |
| 33M | dulaglutide | 30.03 | 6.70 | 4 | 50.98 | 12.95 | 4 |
| | 270 μg | | | | | | |
| 34M | exenatide (E1) | 31.00 | 7.24 | 4 | 40.27 | 4.83 | 3 |
| | 92.31 μg | | | | | | |
| 35M | exenatide (E2) | 30.33 | 3.74 | 4 | 42.68 | 6.55 | 4 |
| | 70.6 μg | | | | | | |
| 36M | lixisenatide | 33.55 | 3.23 | 4 | 55.15 | 12.74 | 4 |
| | 3 μg | | | | | | |
| 37M | liraglutide | 32.88 | 6.78 | 4 | 63.60 | 9.71 | 4 |
| | 180 μg | | | | | | |

TABLE 22
Summarized averages of right paw withdrawal threshold in grams
measured by von Frey filaments at D2, D7 and D10 (mean ± SD, N)

| | | D2 | | | D7 | | | D10 | | |
|---|---|---|---|---|---|---|---|---|---|---|
| Group | Treatment | Mean | SD | N | Mean | SD | N | Mean | SD | N |
| 31M | Control/Vehicle | 34.50 | 17.00 | 4 | 29.00*** | 21.31 | 4 | 20.50*** | 5.88 | 8 |
| 32M | MIA/Vehicle | 1.60^{$$$} | 0.49 | 4 | 1.70 | 0.35 | 4 | 1.38 | 0.31 | 8 |
| 33M | dulaglutide | 2.28^{$$$} | 0.30 | 4 | 2.85 | 1.35 | 4 | 4.75*** | 1.49 | 8 |
| | 270 μg | | | | | | | | | |
| 34M | exenatide (E1) | 1.45^{$$$} | 0.41 | 4 | 3.35 | 2.09 | 4 | 4.77** | 1.76 | 7 |
| | 92.31 μg | | | | | | | | | |
| 35M | exenatide (E2) | 1.55^{$$$} | 0.30 | 4 | 5.00* | 2.58 | 4 | 5.25*** | 2.60 | 8 |
| | 70.6 μg | | | | | | | | | |
| 36M | lixisenatide | 1.30^{$$$} | 0.20 | 4 | 5.00** | 1.15 | 4 | 7.63*** | 3.11 | 8 |
| | 3 μg | | | | | | | | | |
| 37M | liraglutide | 1.60^{$$$} | 0.49 | 4 | 8.00*** | 0.00 | 4 | 9.63*** | 2.62 | 8 |
| | 180 μg | | | | | | | | | |

TABLE 22bis
Summarized averages of right paw withdrawal threshold in grams
measured by von Frey filaments at D18, D25 and D31 (mean ± SD)

| | | D18 | | | D24 | | | D30 | | |
|---|---|---|---|---|---|---|---|---|---|---|
| Group | Treatment | Mean | SD | N | Mean | SD | N | Mean | SD | N |
| 31M | Control/Vehicle | 34.63*** | 21.50 | 8 | 26.13*** | 14.73 | 8 | 27.50*** | 14.04 | 8 |
| 32M | MIA/Vehicle | 2.18 | 0.77 | 8 | 2.18 | 0.77 | 8 | 1.93 | 0.21 | 8 |
| 33M | dulaglutide | 6.50*** | 0.93 | 8 | 7.00*** | 1.07 | 8 | 8.25*** | 0.71 | 8 |
| | 270 μg | | | | | | | | | |
| 34M | exenatide (E1) | 4.86*** | 1.07 | 7 | 5.43*** | 1.51 | 7 | 6.29** | 2.14 | 7 |
| | 92.31 μg | | | | | | | | | |
| 35M | exenatide (E2) | 6.00*** | 1.85 | 8 | 6.75*** | 1.04 | 8 | 7.25*** | 1.04 | 8 |
| | 70.6 μg | | | | | | | | | |
| 36M | lixisenatide | 7.75*** | 1.83 | 8 | 7.25*** | 1.04 | 8 | 8.88*** | 1.67 | 8 |
| | 3 μg | | | | | | | | | |
| 37M | liraglutide | 11.63*** | 2.88 | 8 | 10.25*** | 3.06 | 8 | 9.00*** | 3.06 | 8 |
| | 180 μg | | | | | | | | | |

For tables 22 and 22bis:
^{$$$} p<0.001 Control/vehicle group (31M) vs MIA/vehicle group and treated groups (32M, 33M, 34M, 35M, 36M and 37M) at day 2.
* p<0.05 MIA/vehicle group (32M) vs exenatide (E2)-treated group (35M) at day 7.
** p<0.01 MIA/vehicle group (32M) vs lixisenatide-treated group (36M) at day 7.
** p<0.01 MIA/vehicle group (32M) vs exenatide (E1)-treated group (34M) at day 10 and 30.
**p<0.001 MIA/vehicle group (32M) vs liraglutide-treated group (37M) at day 7, 10, 18, 24 and 30.
*** p<0.001 MIA/vehicle group (32M) vs lixisenatide-treated group (36M) at day 10, 18, 24 and 30.
*** p<0.001 MIA/vehicle group (32M) vs exenatide (E2)-treated group (35M) at day 10, 18, 24 and 30.
*** p<0.001 MIA/vehicle group (32M) vs exenatide (E1)-treated group (34M) at day 10, 18 and 24.
*** p<0.001 MIA/vehicle group (32M) vs dulaglutide-treated group (33M) at day 10, 18, 24 and 30.
*** p<0.001 MIA/vehicle group (32M) vs Vehicle/control group (31M) at day 2, 7, 10, 18, 24 and 30.
Until day 7, mean±SEM, n=4 per group.
From day 10, mean±SEM, n=7-8 per group.

### Conclusion

In average, all groups have responses within the normal range (within 15-50 g range) using the von Frey filaments for the left (healthy) hind paw at each tested time point. There was not any difference between groups for the left hind paw.

For the right hind paw, MIA IA injection on day 1 resulted in a significant reduction on paw withdrawal threshold that was characterized on day 2 between saline group (31M) and the rest of the MIA groups (32M-37M) (mean±SD; $p<0.005, ^{$$$}p<0.001). For group 32M, this effect lasted until study termination thus, validating the MIA IA injection and MIA rat model on the rapid pain-like responses in the ipsilateral limb in rats (FIG. 18).

Comparing only 4 animals per group and using the von Frey filaments technique on day 7, results indicated that there was a significant increase of paw withdrawal threshold in MIA-injected animals treated with 70.6 μg of exenatide (E2), 3 μg of lixisenatide and 180 μg of liraglutide (35M, 36M and 37M) compared to MIA/vehicle treated group (32M) (mean±SD; *p<0.05, **p<0.01, ***p<0.001) (FIG. 18).

Upon day 10 until day 30, all treated groups (33M, 34M, 35M, 36M and 37M) displayed a significant increase of paw withdrawal threshold compared to MIA/vehicle treated group (32M) for each day (mean±SD; **p<0.01, ***p<0.001) (FIG. 18).

### Comparative Test: Von Frey Filaments Vs Electronic Von Frey

Allodynia in rats following OA induction on day 1 was evaluated on day 2 and on day 7 using both Electronic (for cycle 1) and von Frey filaments (for cycle 2). Both tests are reliable and valid for the assessment of the mechanical sensitivity in the ipsilateral hind paw of rats. Additionally, von Frey filament score can be transposed approximately to an equivalent electronic von Frey score (theorical pressure in grams/mm²). To assure the obtained results, we performed a test using both von Frey filaments and Electronic von Frey apparatus on three rats from three different groups (31M, 32M and 37M). Equivalent electronic von Frey score transposed from von Frey filament score was found comparable to the mean score using electronic von Frey apparatus on the same animals.

Equivalent electronic von Frey score was calculated using the “Aesthesio Precision Tactile sensory data chart” from the results obtained with von Frey filaments. Thus, to have a more representative vision of the results obtained in previous experiments using the electronic von Frey apparatus, we combine both the electronic von Frey scores obtained from Cycle 1 (Day 2 and 7) and the equivalent score calculated from the results obtained with the von Frey filaments for Cycle 1 (from day 10) and Cycle 2. Additionally, to have an idea of the paw withdrawal effect in rats of the different treatments compared to the vehicle group, we normalized the previous results (electronic equivalent score from von Frey filaments+the electronic von Frey score from Cycle 1 at day 2 and 7) to their correspondent vehicle group (%) from Cycle 1 or Cycle 2 at each day. When von Frey scores were normalized to vehicle group (31M), liraglutide/MIA treated group (37M) reached a score of 70% at day 10 and maintained this level for 20 days. Similarly, lixisenatide/MIA treated group (36M) raised to 60% and kept it. dulaglutide/MIA- (33M), exenatide (E1)/MIA- (34M), exenatide (E2)/MIA-(35M) treated groups gradually reached a 50-60% score at day 30 when compared to the vehicle group (31M) that were normalized to 100%.

### Conclusion

In conclusion, thanks to this comparative study between IA injection of solutions comprising 180 μg of liraglutide, 270 μg of dulaglutide, 92.31 μg of exenatide (E1), 70.6 μg of exenatide (E2) and 3 μg of lixisenatide, we observed:
All GLP-1 analogues show an increasing anti-inflammatory/analgesic effect during 30 days with a similar profile.
Von Frey filaments assay in rats can be translated to analogous electronic von Frey score with the “Aesthesio Precision Tactile sensory data chart” with the purpose of comparing different experiments that used divergent but complementary methods.

In conclusion, this study confirms that locally-administration (IA administration) of solutions comprising 180 μg of liraglutide, 270 μg of dulaglutide, 92.31 μg of exenatide (E1), 70.6 μg of exenatide (E2) and 3 μg of lixisenatide targets relevant mechanisms associated with pain in a MIA-induced OA and inflammatory pain model in rats. Therefore, the IA administration of solutions comprising various GLP-1R agonists, such as liraglutide, dulaglutide, exenatide and lixisenatide, targets relevant mechanisms associated with pain in a MIA-induced OA and inflammatory pain model in rats and thus can be used in a method for treating joint diseases, in particular osteoarthritis and/or joint pain.

### Example 8: Comparison Between the Analgesic Effect of a Composition According to the Prior Art and a Composition According to the Present Invention

### Test System

Sprague Dawley Rat.

### Test Items

TABLE 23
composition according to the invention

| Pre- formulation | Tonicity | | Liraglutide API |
|---|---|---|---|
| name | modifier | Buffer agent | (6 mg/ml) |
| Pre-formulation 2 | Glucose | Tromethamine | yes |
| Pre-formulation | Propylene glycol | Phosphate | yes |
| 11 | | | |

Pre-formulation 2 is identical to pre-formulation 2 of example 1.

TABLE 24
composition according to the prior art

| Hydrogel | |
|---|---|
| formulation | Hydrogel composition (in PBS pH 7.4) + |
| name | 1 mg/mL of liraglutide |
| P6 | Albumin |
| P8 | HPMC, polysorbate 80 |
| P20 | Hyaluronate sodium, poloxamer 407 |

Hydrogel formulations P6, P8 and P20 are identical to formulations No. 6, 8 and 20 respectively disclosed in table 3 pages 23-24 of patent application WO 2020/104833.

### Formulations:

Pre-formulation 2 of liraglutide supplied as solution at 6 mg/ml was diluted in PBS to reach the final concentrations of 180 μg in 30 μL.

Pre-formulation 11 of liraglutide was obtained from Victoza®, which is a commercial liraglutide drug. Test item supplied as a solution at 6 mg/ml (Victoza®) was diluted in PBS to reach the final concentration of 20 μg in 30 μL.

The vehicle (PBS) is supplied “ready to use” and was diluted in cell seeding medium at a final concentration of 1:100.

### Experimental Design

Surgery-induced OA: OA induction by medial ligament transection (MLT) procedure followed by resection of medial menisci (MMx): Groups 41M, 42M, 43M and 44M

Anesthesia was induced for each rat by a chamber induction technique using inhalation anesthesia (Isoflurane at 4.0%). During surgery, the animal was maintained with Isoflurane at a level between 1.5 and 2.5% with an oxygen flow rate of 1-2 liters/minute. Ophthalmic ointment was applied to the eyes to prevent drying of the tissue during the anesthetic period. After induction of anesthesia, the right leg skin surface was clipped free of hair using electric animal clippers. After shaving the knee joint, the skin was disinfected with iodine and a para patellar skin incision was made on the medial side of the joint. An incision on the medial side of the joint space was made. The medial ligament was transected and the medial meniscus was resected using a microsurgical knife. The wound was closed with vicryl 5/0 braided absorbable suture. All operation procedures were performed using a surgical microscope. Group Allocation is show in Table 25 and study timeline in Table 26.

TABLE 25
Group allocation.

| | | | | Dose | Dose | Route of | |
|---|---|---|---|---|---|---|---|
| | | OA | | level | volume | administration | Dose |
| Group | N= | induction | Treatment | (μg) | (μL) | (ROA) | regimen |
| 41M | 11 | √ | Vehicle (PBS) | — | 25 | Intra-articular | Once on |
| 42M | 12 | √ | Formulation | 54 | | | day 7 |
| | | | P6 | | | | |
| 43M | 12 | √ | Formulation | 54 | | | |
| | | | P8 | | | | |
| 44M | 12 | √ | Formulation | 54 | | | |
| | | | P20 | | | | |

TABLE 26
Study timeline for Groups 41M, 42M, 43M and 44M.

| Study | | |
|---|---|---|
| Day/week* | Procedure | Remarks |
| D 1 | OA induction | |
| D 7 | IA Treatment (Vehicle, P6, P8 or | IA single injection, right knee |
| | P20 hydrogel formulation) | |
| D −1, D 14, | Incapacitance test for all groups | |
| D 28, D 35 | | |
| D 35 | Termination | Right knee collection and fixation |
| | | Left knee collection and fixation |

### OA Induction by Intra-Articular (IA) Injection of MIA (Monosodium Iodoacetate): Groups 45M, 46M, 47M and 48M

The protocol for OA induction by intra-articular (IA) injection of MIA (monosodium iodoacetate) for groups 46M, 47M and 48M was as disclosed in example 6; animals were injected MIA once on day 1. For group 45M (sham control), 30 μL of injectable saline was injected into knee joint.

### Mechanical Pain Evaluation (Von Frey Test): Groups 45M, 46M, 47M and 48M

The protocol for mechanical pain evaluation was as disclosed in example 6.

TABLE 27
Group allocation

| | | MIA | | | | | |
|---|---|---|---|---|---|---|---|
| | | induction | | | | | |
| | | (3 mg in | | Dose | Dose | | Dose |
| Group | N= | 30 μL) | Treatment | level | volume | ROA | regimen |
| 45M | 8 | — | Vehicle (PBS) | — | 30 μL | IA | Once on |
| 46M | 8 | √ | Vehicle (PBS) | — | | | day 3 |
| 47M | 8 | √ | Pre-formulation 2 | 180 μg | | | |
| 48M | 8 | √ | Pre-formulation 11 | 20 μg | | | |

N = number of animals.

### Statistical Plan for Groups 45M, 46M, 47M and 48M

TABLE 28
Study timeline for groups 45M, 46M, 47M and 48M

| Study | | |
|---|---|---|
| Day/week* | Procedure | Remarks |
| D 1 | MIA injection, 3 mg in 30 μL, IA | Except for group 45M (sham |
| | in the right knee | control): 30 μL injectable saline, IA |
| D 3 | IA Treatment (Vehicle, Pre- | IA single injection, 30 μL, right knee |
| | formulation 11, or Pre- | |
| | formulation 2) | |
| D 2, D 7, D 10, | Von Frey test for all groups | |
| D 18, D 25, and | | |
| D 31 | | |
| D 31 | Termination for all groups | Right knee (diseased) collection |

For statistics, we applied a sequential testing strategy reflecting experimental expectations, while retaining the central Mann-Whitney (non-parametric) test. Sequential statistics were made to compare in this order:
1. The control/vehicle group (45M) and the MIA/vehicle group (46M).
2. The MIA/vehicle group (46M) and the MIA/pre-formulation 11-treated animals (20 μg) (48M).
3. The MIA/vehicle groups (46M) and the MIA/pre-formulation 2-treated animals (180 μg) (47M).

### Mechanical Pain Evaluation (Von Frey Test) for Groups 45M, 46M, 47M and 48M

Allodynia in rats following OA induction on day 1 was evaluated on day 2 as baseline for group allocation and then on days 7, 10, 18, 25 and 31 to assess effect of treatments on mechanically-induced pain. For each group, mean of withdrawal responses, as given by the electronic von Frey apparatus, for the left and right hind paws, was calculated. Group average animal withdrawal response results are presented in Table 29 and Table 30 for the right hind paw. Results on the right hind paw are presented in FIG. 20. FIG. 20 compares MIA/vehicle group (46M) to MIA/pre-formulation 11 (48M) and MIA/vehicle (46M) to the group of Preformulation-2 (47M).

On day 7, day 10, day 18, day 25 and on day 31 there was a significant increase of paw withdrawal threshold in MIA-injected animals between pre-formulation 11-treated group 48M vs MIA/vehicle group 46M (mean±SD; p<0.001). A significant dose effect and a significant increase of paw withdrawal threshold in MIA-injected animals was observed in preformulation-2 group (47M) vs MIA/vehicle group 46M (mean±SD; p<0.001, p<0.01 and p<0.05) on day 7, day 10, day 18, day 25 and on day 31.

Statistical analysis revealed significant differences between the control group and the treated groups (47M, 48M) with liquid formulations according to the invention (pre-formulation 11 and pre-formulation 2) up to 4 weeks after single IA injection on the right knee. There is no significant difference between the paw withdrawal threshold of pre-formulation 11-treated group 48M and pre-formulation 2-treated group 47M. The analgesic effect was maintained up to 4 weeks following the single intra-articular injection of pre-formulations 2 and 11 according to the invention, as it can be seen in FIG. 20 with the curve plateau which is stable up to day 31.

TABLE 29
Summarized averages of right paw withdrawal threshold
in grams at D2, D7 and D10 (mean ± SD)

| | | D2 | | D7 | | D10 | |
|---|---|---|---|---|---|---|---|
| Group | Treatment | Mean | SD | Mean | SD | Mean | SD |
| 45M | Control/Vehicle | 85.80*** | 8.43 | 91.59*** | 7.88 | 94.16*** | 3.96 |
| 46M | MIA/Vehicle | 28.08 | 5.49 | 25.55 | 3.26 | 25.40 | 4.15 |
| 47M | Preformulation 2 | 27.96 | 2.83 | 59.99*** | 5.98 | 62.46*** | 5.51 |
| | (180 μg) | | | | | | |
| 48M | Preformulation | 27.70 | 3.48 | 73.43*** | 6.04 | 78.95*** | 7.87 |
| | 11 (20 μg) | | | | | | |

TABLE 30
Summarized averages of right paw withdrawal threshold
in grams at D18, D25 and D31 (mean ± SD)

| | | D18 | | D25 | | D31 | |
|---|---|---|---|---|---|---|---|
| Group | Treatment | Mean | SD | Mean | SD | Mean | SD |
| 45M | Control/Vehicle | 92.14*** | 10.16 | 95.71*** | 5.99 | 89.39*** | 4.96 |
| 46M | MIA/Vehicle | 30.68 | 4.89 | 37.85 | 9.39 | 38.91 | 7.50 |
| 47M | Preformulation | 56.61*** | 8.53 | 60.28*** | 5.87 | 65.43*** | 9.50 |
| | 2 (180 μg) | | | | | | |
| 48M | Preformulation | 78.75*** | 5.83 | 76.51*** | 8.41 | 72.99*** | 9.65 |
| | 11 (20 μg) | | | | | | |

MIA/Vehicle (46M) vs Control/Vehicle (45M) (Mann-Whitney test; ***p < 0.001).
MIA/Vehicle (46M) vs MIA preformulation-2 and preformulation 11 (47M and 48M) (Mann-Whitney test; *p < 0.05, **p < 0.01, ***p < 0.001).

### Mechanical Pain Evaluation (Incapacitance Test) for Groups 41M, 42M, 43M and 44M

Weight-bearing changes in the rats with OA were measured using an incapacitance tester. Postural imbalance, which reportedly indicates a change in the pain threshold and weight distribution of the limbs, is decreased. Each rat was placed so that each hind paw rested on a separate force plate on the incapacitance apparatus, and the weight borne by each hind limb was measured for 5 s. The ratio of the weight borne by the right to left hind limb is calculated. The mean of 5 consecutive measurements for each rat was recorded. Weight bearing function (Incapacitance test) was performed at baseline (Day −1), day 14, day 28 and day 35: total of four times. The experimenter(s) were blind regarding to the groups.

Rats were sacrificed via CO₂ asphyxiation on Day 36. The knee articular structure was fixed in 4% buffered formalin solution for further histological analysis. Contralateral (non-injured) knees were also fixed in 4% buffered formalin solution.

Numerical results were given as means±SD. Outliers data points (marked with asterisk) were identified following Grubbs' test analysis with alpha=5% and were not included in the group average calculations. If applicable, statistical analysis was carried out using two-way (followed by Bonferroni post-hoc test) or one-way ANOVA (followed by Dunnett's Multiple Comparison post Test). A probability of 5% (p<0.05) was regarded as significant. In the figures, the degree of statistically significant differences between groups were illustrated as *p<0.05, **p<0.01 and ***p<0.001.

The results are presented in FIG. 19. Statistical analysis revealed significant difference between the control group 41M and the treated group 42M with gel formulation P6 only on day 14, e.g. 7 days after single IA injection on the right knee. There was no significant difference between the control group 41M and the treated group 42M with gel formulation at days 28 (e.g. 14 days after single IA injection on the right knee) and 35 (e.g. 21 days after single IA injection on the right knee). Thus, the effect induced by formulation P6 according to the prior art was significant 7 days after single IA injection on the right knee but not starting to 14 days after single IA injection on the right knee. This can be seen in FIG. 19, where the effect obtained on day 14 (e.g. 7 days after single IA injection on the right knee) is not maintained afterwards (i.e. on days 28 (e.g. 14 days after single IA injection on the right knee) and 35 (e.g. 21 days after single IA injection on the right knee)). Furthermore, there was no significant difference between the control group 41M and the treated groups 43M and 44M, which explains why there is no curve related to groups 43M (formulation P8) and 44M (formulation P20) in FIG. 19.

### Conclusion

In conclusion, in this comparative study between IA injection of solutions versus gel formulations, we observed that solution formulations according to the invention (groups 47M and 48M) provided an analgesic effect for up to 4 weeks after single IA injection in the right knee, whereas gel formulations according to the prior art (groups 42M, 43M and 44M) provided either no statistically significant analgesic effect or an analgesic effect only on day 14, e.g. 7 days after single IA injection on the right knee.

### Example 9: Comparison of Pharmacokinetic in Synovial Fluid and Plasma Between Three Pre-Formulations According to the Present Invention

### Test System

Dogs, Beagles, 3 to 6 years

### Test Items

TABLE 31

| Pre- formulation | Tonicity | | Liraglutide API |
|---|---|---|---|
| name | modifier | Buffer agent | (6 mg/ml) |
| Pre-formulation 2 | Glucose | Tromethamine | yes |
| Pre-formulation 6 | PEG400 | Tromethamine | yes |
| Pre-formulation | Propylene glycol | Phosphate | yes |
| 12 | | | |

Pre-formulations 2 and 6 are identical to pre-formulations respectively 2 and 6 of example 1. Pre-formulations 2, 6 and 12 are pharmaceutical compositions according to the present invention.

### Formulations:

Pre-formulation 2: Liraglutide 6 mg/ml, Tromethamine (8 mM) and Glucose (30 mg/ml) in water for injection.

Pre-formulation 6: Liraglutide 6 mg/ml, Tromethamine (8 mM) and PEG400 (60 mg/ml) in water for injection.

Pre-formulation 12: Victoza® as such: Liraglutide 6 mg/ml, disodium phosphate dihydrate 1.42 mg/ml, propylene glycol 14 mg/ml, phenol 5.5 mg/ml in water for injection. Victoza® is a commercial liraglutide drug.

### Method:

Three groups comprising 2 or 3 dogs per group were allocated for the study of the pharmacokinetics in plasma, and three groups comprising 3 dogs per group were allocated for the study of the pharmacokinetics in synovial fluid.

TABLE 32
Group allocation

| | | | Dose | Dose | | Dose |
|---|---|---|---|---|---|---|
| Group | N= | IA injection | level | volume | ROA | regimen |
| 51M | 3 | Pre-formulation 12 | 0.9 mg | 150 μL | IA | Once at |
| 52M | 2 | Pre-formulation 2 | 0.9 mg | | | T0 |
| 53M | 2 | Pre-formulation 6 | 0.9 mg | | | |
| 54M | 3 | Pre-formulation 12 | 0.9 mg | | | |
| 55M | 2 | Pre-formulation 2 | 0.9 mg | | | |
| 56M | 2 | Pre-formulation 6 | 0.9 mg | | | |

N=number of animals.

TABLE 33
Study timeline for groups 51M, 52M, 53M, 54M, 55M and 56M

| Study hours | Procedure |
|---|---|
| T0 | IA treatment for groups 51M, 52M, 53M, 54M, 55M and |
| | 56M (pre-formulation 12, pre-formulation 2 or pre- |
| | formulation 6): IA injection in the right knee |
| T0 + 0.17 hours, T0 + 2 hours, | Dosage of liraglutide in plasma for groups 51M, 52M and |
| T0 + 3 hours, T0 + 6 hours, | 53M |
| T0 + 8 hours, T0 + 10 hours, | |
| T0 + 10 hours, T0 + 24 hours | |
| T0 + 0.17 hours, T0 + 8 hours, | Dosage of liraglutide in synovial fluid for groups 54M, |
| T0 + 16 hours, T0 + 24 hours | 55M and 56M |

### Statistical Plan

For statistics, we applied the One-way Anova+Tukey Post test.

### Results:

### Plasma Dosage of Liraglutide for Groups 51M, 52M and 53M

The plasma dosage of liraglutide for groups 51M, 52M and 53M is presented in FIG. 21. The Turkey's multiple comparisons test allows to conclude that there is no significant difference of AUC of liraglutide plasma concentration during the 24 hours following the IA injection of pre-formulation 12 versus pre-formulation 2, pre-formulation 12 versus pre-formulation 6 and pre-formulation 2 versus pre-formulation 6.

The measured AUC(=area under the curve) are summarized in the following table:

TABLE 34

| | IA pre- | IA pre- | IA pre- |
|---|---|---|---|
| | formulation | formulation | formulation |
| AUC | 12 | 2 | 6 |
| Total area | 1833 | 792.1 | 1530 |
| Std. Error | 387.2 | 26.91 | 146.4 |
| 95% Confidence | 1074 to 2592 | 739.3 to 844.8 | 1243 to 1817 |
| interval | | | |

### Synovial Fluid Dosage of Liraglutide for Groups 54M, 55M and 56M

The synovial fluid dosage of liraglutide for groups 54M, 55M and 56M is presented in FIG. 22.

The measured AUC(=area under the curve) are summarized in the following table:

TABLE 35

| | IA pre-formulation | IA pre-formulation | IA pre-formulation |
|---|---|---|---|
| AUC | 12 | 2 | 6 |
| Total area | 6223787 | 3699329 | 3335996 |
| Std. Error | 617346 | 916306 | 366909 |
| 95% Confidence | 5013811 to | 1903403 to | 2616866 to |
| interval | 7433762 | 5495254 | 4055125 |

### Conclusion

In conclusion, there is a similar AUC of liraglutide plasma concentration during the 24 hours following an IA injection or a sub-cutaneous injection of liraglutide. However, when liraglutide is administered by sub-cutaneous route, there is no liraglutide in the synovial fluid during the 24 hours following the intra-cutaneous injection, whereas when liraglutide is administered by intra-articular route, there is liraglutide in the synovial fluid during the 24 hours following the intra-articular injection.

### Example 10: Clinical Trial Phase 1

A Phase I Clinical Trial to Evaluate the Safety, Tolerability, Pharmacokinetics and Efficacy of single ascending doses of 4P004 versus placebo injected in the Target Knee Joint of Patients with Osteoarthritis (stage Kellgren-Lawrence (KL) 2-4). 4P004 refers to a composition according to the present invention, that is a composition that comprises GLP-1R agonist for IA injection.

### Objectives

### Primary Objective

To assess the clinical and biological safety, and general and local tolerability of liraglutide when administered as knee intra-articular (IA), single ascending dose, in patients with knee osteoarthritis.

### Secondary Objectives

To determine the plasma PK of liraglutide when administered as single IA doses at escalating dose levels in patients with Osteoarthritis (stage KL 2-4).

### Endpoints

### Primary Endpoints

1. Difference between 4P004 treated subjects and placebo in the number of Adverse Events [Time Frame: from screening until final follow-up visit]
2. Difference between 4P004 treated subjects and placebo in the number of Abnormal vital signs [Time Frame: from screening until final follow-up visit]
3. Difference between 4P004 treated subjects and placebo in the number of Abnormal clinical laboratory evaluations [Time Frame: from screening until final follow-up visit]
4. Difference between 4P004 treated subjects and placebo in the number of abnormal physical examination [Time Frame: from screening until final follow-up visit]

### Secondary Endpoints

Evaluation of PK parameters: (Cmax, Tmax, AUC0-t, AUC0-∞, T½) Day 1 and Day 2.

### Exploratory Endpoints:

Collection of biological fluids (urine, blood, synovial fluids) to evaluate liquid biomarkers associated with 4P004 action.

### Overall Design

This phase I is a randomized, double-blind, placebo-controlled study to assess the safety and tolerability of single ascending dose of intra-articular 4P004 at 0.3 mg, 1 mg, 3 mg, and 6 mg in patients
between 18 and 80 years of age,
with knee osteoarthritis,

A total of 32 participants will be enrolled in 4 cohorts, each cohort will receive either 4P004 or placebo (6:2). 4P004 dose will increase with cohort 1 to 4.

### Inclusion Criteria

Patients who have the capacity to give informed consent and who are willing to comply with all study related procedures and assessments (consent via legally authorized representative will not be accepted)
Patients must be ≥18 and ≤80 years of age
Patients diagnosed with primary osteoarthritis of the knee (stage KL 2-4) assessed locally.

### Exclusion Criteria

Treatment with systemic glucocorticoids greater than 10 mg prednisone or the equivalent per day within 4 weeks prior to screening
Any known active infections
Any chronic condition that has not been well controlled for a minimum of 3 months
History of malignancy of any organ system (other than localized basal cell carcinoma of the skin or in-situ cervical cancer) within the last 2 years
Diabetes type I and type II patients managed by GLP-1 analogues
Patients exposed to Glucagon-peptide 1 analog hormones
Treatment of the target knee with intra-articular injection (steroids, hyaluronic acid derivatives . . . ) within 3 months
Use of topical analgesic agents (gels, creams, or patches) for the treatment of knee OA within 7 days of screening
Effusion of the target knee requiring aspiration within 3 months
Use of electrotherapy or acupuncture for OA within 4 weeks
Significant and clinically evident misalignment of the target knee
Any condition, including laboratory findings, that in the opinion of the investigator constitutes a risk or contraindication for participation in the study or that could interfere with the study objectives, conduct or evaluation
Participation in a clinical research trial within 12 weeks prior
Hypersensitivity to the active substance or to any of the excipients: Disodium phosphate dihydrate, Propylene glycol, Phenol.

### Results

We expect to demonstrate that 4P004 is safe and well tolerated in human for a dose within the tested doses. And we expect to determine the pharmacokinetics parameters of a single intra-articular injection the patients knee joint.

### Example 11: Clinical Trial Phase 2

### Phase II Study

Dose ranging study to assess the efficacy and safety of 4P004 administered via intraarticular injection in patients with mild to moderate osteoarthrosis of the knee. 4P004 refers to a composition according to the present invention, that is a composition that comprises GLP-1R agonist for IA injection.

### Objectives

### Primary Objective

To demonstrate the efficacy of 4P004 in knee osteoarthritis and to determine the optimal dose regimen

### Secondary Objectives

To assess the safety and tolerability of 4P004 different dose regimen versus placebo

To assess complementary clinical efficacy of 4P004 versus placebo

### Exploratory Objectives

Biomarkers

Clinical signs of structural change by imaging

### Endpoints

### Primary Endpoint

To demonstrate significant pain improvement using Western Ontario and McMaster Universities Osteoarthritis Index (WOMAC) pain score of the knee versus placebo at 3 months

Change from baseline in OMERACT-OARSIS score

### Secondary Endpoints

Clinical and laboratory safety
Change from baseline pain using a Visual Analog Scale (VAS) For phase II study participants
Change from baseline per physician global assessment of disease scale
Change from baseline patient global assessment (PGA)
Change from baseline in OMERACT-OARSIS score
Change from baseline in MRI-assessed synovitis at 6 and 12 months
Change in joint space narrowing in the target knee.

### Inclusion Criteria

Male or female between 18 and 80 years of age,
Using contraceptive consistent with local regulations regarding the methods of contraception for those participating in clinical studies,
Willing and able to provide written informed consent
Established Clinical diagnosis of OA in the target knee for at least 6 months (clinical and x-ray criteria)
Radiographic (x-ray) disease Stage 2 or 3 in the target knee according to the Kellgren-Lawrence grading of the knee OA
Primary source of pain throughout the body is due to OA in the target knee
pain visual analog scale (VAS) score of 30-80 mm (on 100-mm VAS) and a WOMAC total
score of 72-192 (of 240) for the target knee at screening
ambulatory; assistive devices (e.g., canes) were allowed if needed <50% of the time, whereas any use of a walker was excluded
Body mass index <40

### Exclusion Criteria

Major knee surgery in the target knee within 12 months prior to study or planned surgery during the study period
Partial or complete joint replacement in the target knee
Currently requires regular use of ambulatory assistive devices (e.g wheelchair, parallel bars, walker, canes or crutches)
Comorbid conditions that could affect study endpoint assessments of the target knee, including, but not limited to, Rheumatoid arthritis, psoriatic arthritis, systemic lupus erythematosus, gout or pseudogout, and fibromyalgia.
Diabetes type I and type II patients
Patients exposed to Glucagon-peptide 1 analog hormones
History of malignancy within the last 3 years
Participation in a clinical research trial within 12 weeks prior
Treatment of the target knee with intra-articular steroids within 2 months or hyaluronic acid derivatives within 6 months
Treatment with systemic glucorticoids greater than 10 mg prednisone or the equivalent per day within 4 weeks prior to screening
Effusion of the target knee requiring aspiration within 3 months
Use of electrotherapy or acupuncture for OA within 4 weeks
Significant and clinically evident misalignment of the target knee
Any known active infections
Any chronic condition that has not been well controlled for a minimum of 3 months
Use of centrally acting analgesics (e.g duloxetine) within 12 weeks prior to screening
Use of anticonvulsants within 12 weeks prior to screening, unless used for seizure or migraine prophylaxis
Use of topical analgesic agents (gels, creams, or patches) for the treatment of knee OA within 7 days of screening
Any condition, including laboratory findings, that in the opinion of the investigator constitutes a risk or contraindication for participation in the study or that could interfere with the study objectives, conduct or evaluation

### Overall Design

This is a dose range finding, randomized, double-blind and placebo-controlled study to assess the efficacy and safety 3 dose of intra-articular 4P 004 at 3 ascending doses or placebo in patients
between 18 and 80 years of age,
with mild or moderate knee osteoarthritis,

Approximately 500 participants will be randomized in one of the 4 treatment groups to receive one of the 3 fixed doses of 4P004 or placebo.

### Results

We expect to demonstrate that 4P004 significantly reduced pain compared to Placebo based on the WOMAC assessment test and to obtain clinical signs of function improvement in osteoarthritic patients

1. A method of treating at least one joint disease by administering to a patient in need thereof an effective amount of a pharmaceutical composition,
wherein said pharmaceutical composition is a solution or a suspension to be administered via intraarticular injection, in particular via intraarticular injection into the joint cavity, and wherein said pharmaceutical composition comprises:
a GLP-1R agonist,
a buffer selected from the group consisting of a tromethamine buffer and a phosphate buffer, and
an isotonic agent selected from the group consisting of glucose, a polyethylene glycol, propylene glycol and glycerol.

2. The method according to claim 1, wherein
the GLP-1R agonist is selected from the group consisting of liraglutide, exenatide, lixisenatide, albiglutide, beinaglutide, dulaglutide, semaglutide, pegapamodutide, taspoglutide and combinations thereof,
the buffer is a phosphate buffer, and
the isotonic agent is propylene glycol.

3. The method according to claim 1, wherein said pharmaceutical composition comprises:
a GLP-1R agonist,
a buffer selected from the group consisting of a tromethamine buffer and a phosphate buffer, and
an isotonic agent selected from the group consisting of glucose, a polyethylene glycol and glycerol.

4. The method according to claim 1, wherein the GLP-1R agonist is selected from the group consisting of liraglutide, exenatide, lixisenatide, albiglutide, beinaglutide, dulaglutide, semaglutide, pegapamodutide, taspoglutide and combinations thereof.

5. The method according to claim 4, wherein the GLP-1R agonist is liraglutide.

6. The method according to claim 4, wherein the GLP-1R agonist is semaglutide.

7. The method according to claim 19, wherein said dose of said pharmaceutical composition is to be administered in one or at least two intraarticular injections.

8. The method according to claim 1, wherein doses of said pharmaceutical composition are to be administered every month.

9. The method according to claim 1, wherein the total dose of GLP-1R agonist that is administered in one year is from 0.18 mg to 72 mg.

10. A pharmaceutical composition comprising:
a GLP-1R agonist,
a buffer selected from the group consisting of a tromethamine buffer and a phosphate buffer, and
an isotonic agent selected from the group consisting of glucose, a polyethylene glycol and glycerol,
wherein said pharmaceutical composition is a solution or a suspension.

11. The pharmaceutical composition according to claim 10, wherein the GLP-1R agonist is selected from the group consisting of liraglutide, exenatide, lixisenatide, albiglutide, beinaglutide, dulaglutide, semaglutide, pegapamodutide, taspoglutide and combinations thereof.

12. The pharmaceutical composition according to claim 10, wherein the pharmaceutical composition comprises from 2 mg/mL to 20 mg/mL of GLP-1R agonist.

13. The pharmaceutical composition according to claim 10, wherein the pharmaceutical composition comprises from 0.01 mg/mL to 20 mg/mL of GLP-1R agonist.

14. The pharmaceutical composition according to claim 10, wherein the buffer is a tromethamine buffer comprising tromethamine as buffering agent.

15. The pharmaceutical composition according to claim 10, wherein the buffer is a phosphate buffer comprising disodium phosphate as buffering agent.

16. The pharmaceutical composition according to claim 10, wherein the isotonic agent is glucose.

17. The pharmaceutical composition according to claim 10, wherein the isotonic agent is a polyethylene glycol having a molecular weight being less than 800 g·mol⁻¹.

18. The pharmaceutical composition according to claim 10, wherein the isotonic agent is glycerol.

19. The method according to claim 4, wherein the GLP-1R agonist is liraglutide and said pharmaceutical composition is to be administered at a dose ranging from 0.0245 mg to 6.3 mg of liraglutide.

20. The method according to claim 4, wherein the GLP-1R agonist is semaglutide and said pharmaceutical composition is to be administered at a dose ranging from 0.0245 mg to 6.3 mg of semaglutide.
